SEQUENCE LISTING (48 sequences)

SEQ ID NO: 1 - DNA, 231 bp, Homo sapiens
gcctcgggcaaactctttggacagacagaccttccaagagggccctcaggctccgcacgtggtctgggcgggaagtgcgggcaagagactaggaagccttcccagcagcgccggcggaccggggcaaggaaagggtcgaatagttaccatgccattctggggccgtccctctgattggcccgagctccctcagcccgagtcgcggcgctccaaggaagggggcggagtctc

SEQ ID NO: 2 - DNA, 21 bp, Artificial sequence
  Primer
ccagtggcccattcagaagtc

SEQ ID NO: 3 - DNA, 20 bp, Artificial sequence
  Primer
ccacttctcgggagccagag

SEQ ID NO: 4 - DNA, 1018 bp, Homo sapiens
ggatacacagtgtagcgatgcatcactctgaaatattttagtttctttttttcccctaaatctgggtatgttcgtgggaatttgcagcacatgtgaacaacttctgtcattcttgcatgaggcaaagggaattgaaaaccacgattactttagaaaactagtttcacagattggtcactgtataaaagaaggatattggttttggtagcttgtgaccacacaccatttctgatctgaataaattcagaacttataatacagttcagaaattgaatgcagtttctcaatatgaggaaagtattttagaataaggcctatttttcaaaggatctgtggaaatcaatgctatgctctcatttaggagatggaaagagtgaggttaaattatcatttcgattaaatctacagtccagattactggtggatgaattgaatgtacttttttattcatataaaacatttgaaatcagaaatctggagtacttttaaatcccattatttattttgttttaatcgccaggtaattcctgagacaggagtgtcccgaagagcctttgcaattatgtaagaatctccgaggcagttcttatgttcctcaattcaaaagaaccacataactgcaatttaaataacaccccacacacacacaaaataaggtcgaagtttatctcaaaataatttcccctctctacactgggataaatatgtataggaataatagggggaaattcagtgcactgagcattaagctgtcaaaacaggaatgtttaaaatatcctgttagtggtttaaaaataatttgtactctaagtccagtgactatttgccagggagaaccaaagttgagaaatttctattaaaaacatgactcagagaaaaaaatgcagaggccggtaatgaaggaaatgattggatctcattcccaattggtcattcctaagatcacatgttctgagcatctttaaaaggaagttatctggactcaagagggtcacagcaccctcctgaaaactgcagc

SEQ ID NO: 5 - DNA, 22 bp, Artificial sequence
  Primer
ggatacacagtgtagcgatgca

SEQ ID NO: 6 - DNA, 21 bp, Artificial sequence
  Primer
gctgcagttttcaggagggtg

SEQ ID NO: 7 - DNA, 1047 bp, Homo sapiens
ggtagcagtctcctggaggtttctctaagcctgagtgaatgaatgaatgaatgaatgaataattgaaacgatagaatcaaaaatgtactttaggatgtatggttgaaaaccacaaacaatgctgaagaagaacctgccttcttcatgacggtgttggaggagttcccggaatgttttcttggctcaaattattacccagcagtggccaccctcagattccagcaaaccagtctcaagttttcactgtttaactctgaattttcttggcagcctaagaggtgagagtatgtggtaataatacatgtataggagttaatgggaagaggaagaattcaagaactaatatttactgaaaacttcctagtgatccttcctcaatgctagtccctttcaatattttatatctttaaccctccttatagtcccatgaaatgattatctccattttttcgttaatgaaatggaacatcagagaaatacaatgttcacagtcacactccggttggtgatggacatgaatatctacaccaaggactaaaatgaaatcatgcctggaagccagctgggtgaaggccctgggaacccatgaactggccatgaaaccagaggatgtcactgacagggaggaccggctgggagctaaatcactcttcagctctttggctgtgagactgcatttgatcaaaaccagaaattaggcctcagacttgtttaactgtagctagaagatccaaattctttcaagagacagagattgtttatcccttgcttcttttggaattctgtattctaactctatggggtgcattttgttttataagctggaagaagagatgttgctgcattaattttgcaatatggaaggagctagcatttgttcaacatcagtcacacactgatcattcttctaaacttctctttgttttatcctacaaaaattacctaaggttaatgtggttgttattctcattttacatttgaggatactgaggtttttaaagtaacttgctcagagtaaatgatagatctgggatccagggtcactgc

SEQ ID NO: 8 - DNA, 21 bp, Artificial sequence
  Primer
ggtagcagtctcctggaggtt

SEQ ID NO: 9 - DNA, 20 bp, Artificial sequence
  Primer
gcagtgaccctggatcccag

SEQ ID NO: 10 - DNA, 736 bp, Homo sapiens
ccttcctctgtgccaagggaagaaaacaccatgcttccctctcctgagagaccttcaagagtttagagacgcctaagtccagctgtgctaaaagataaaggacaataatgcaagtctgctctgagctgccaccatagccagacatagggtagccctgaaagactagaaccaaggacagagccaaaggtgaaagaaaatatgaaaaagtgaaaacacagtatttaaacagaattttccaacagcctgcatatagtgaggacttctcaagcttctgaatccttttccattgaattgtgcaatggcacatgtatgagcaaagtcaagcctcctggctcccaggtaggcacagcccagttcttagctcctaggaagcttcagggcttaaagctccactctacttggactgtactatcaggcccccaaaatggggggagccgacagggaaggactgatttccatttcaaactgcattctggtactttgtactccagcaccattggccgatcaatatttaatgcttggagattctgactctgcgggagtcatgtcaggggaccttgggagccaatctgcttgagcttctgagtgataattattcatgggctcctgcctcttgctctttctctagcacggtcccactctgcagactcagtgccttattcagtcttctctctcgctctctccgctgctgtagccggaccctttgccttcgccactgctcagcgtctgcacatcc

SEQ ID NO: 11 - DNA, 21 bp, Artificial sequence
  Primer
ccttcctctgtgccaagggaa

SEQ ID NO: 12 - DNA, 20 bp, Artificial sequence
  Primer
ggatgtgcagacgctgagca

SEQ ID NO: 13 - DNA, 919 bp, Homo sapiens
gggtgataacacactcagcctggtcaactgaacactttctcccgggctcgggtacacctggagctacaccggcagcccgcggcagtcagagagcatgtaggggcgcgagaggaaaggcaggagagagagaatgctgcagaacaacagtttagggcgtggaaagactaaccaaataaagacccagagctaaaaagctactgagggtctacactcctgggtatttccaaacatctccctcatacccccccactcatcccactcagatgagcctcttctctgaagctcagattcagcagtctctttctagaaatgaccatctagaaatgaagttgaatttcacaatgagaaagttgttcccgaaggcgatgggtcgggcaaaccttaagttacagggtttgccttgtcctgtttcttgatgtttggggagctctggagagtaaaggaaagaaatgaagttgcactaaccttcagccgagttacaggcgttttcgaggaaattaaaggtggacagtgtcgtaattcaatgaaggacaaagtttccaagatttttagaaaagcaatggggagtccagcctgtccaatctcctccctgaaatacagacacaggaagcttcagggtttcttcccgacagagattcagctgggcatatgtagactcaccaggcaggcccttccatgccttccctgtttgtctcattggaaatgagtgggaagcctttaccaaacatagcacttaaaatgaatgtataaaagaaatgacttgaaacaacagaaaataccacccattgcactgtgtaaatccttgcagagagaagatcctgcaagaggaaagctagtccatgaactcatttaacatttatgatgtaatgaactggcccctttgctacagtgtaggtggagaggtcatttccatcttagg

SEQ ID NO: 14 - DNA, 21 bp, Artificial sequence
  Primer
gggtgataacacactcagcct

SEQ ID NO: 15 - DNA, 22 bp, Artificial sequence
  Primer
cctaagatggaaatgacctctc

SEQ ID NO: 16 - DNA, 738 bp, Homo sapiens
ggcaacctcagagttgggagtgaagggaagagcttccagatggatattgctgtggccaggcctcggtgctgccttgctctggggacggctggaggctcgggctcctggagcccgctcccactgcacgcagccctccgcgtctgaggcagcacagcagagtaacgaacggcccggctcgctcatggcaatgacatcaccacaaagactgacacaagctgaagctatttttttttctccaagccttttatctctaggcagtgcagtggagcaaattgaacatgattatgtgctaaatctgaactcagactaaatcaattcaagcagcgttagctaggaactgagtcatagctgttgttgcagccgagtgcttatgtttgcaaaaagcaggagggggtgaatctgacaccagagtttcttctttgaggtgggggaggtgtaattctgcagatgagcctcctgaggttaaggtttgacaattttctgccttcgagatgagcaggaagttgaggcattttgcaaattgcttggcttctgttaattgctctgtgccactcagagcagccacacatgttctgggcatcctaatgcatcccgggcatgggctgaatagaaatccgttcttggagtgactaaagagctggtcgtctgtcatttagggagcatggtaagaggagataattagaggtttgtggaaattctatttgaaggctataagtgcagaccagtaacgctaagagc

SEQ ID NO: 17 - DNA, 21 bp, Artificial sequence
  Primer
ggcaacctcagagttgggagt

SEQ ID NO: 18 - DNA, 21 bp, Artificial sequence
  Primer
gctcttagcgttactggtctg

SEQ ID NO: 19 - DNA, 959 bp, Homo sapiens
cctctgccttggtatatcccacaggctcggtctagcaacagaagggccaccgcctccctgcaacagggcagctgtgaactgaggctggggaaggggcctgtggcttgtagttgacctcagtgtttgccctgctcagctggggccaattacagccccaaggacagctccaatcgatccctgtagcctggctggggtcagcagtaccaagaggccgggatggctgcttcagaagaggcattggccaagcacaatagggccctggagcaccaggattgggctccgccccccaaaagtcccccacagagggcatgcgaggatggggagcgacctggcctttctgctgagtcatgccatctggacctcacagctctgtgagccagcaggtcaagcctgattgggcccatttctcacaggaaaaaactgaggcccaaggagaggaagtgacttgccagagacctcagggaagtctatgggcagagccaagaccagaacccaggtatcctgtctccagagttccttctccagcccccaggcttgccctagcctttgcaaataatagagacattaacaatgatgactgttacgagcttccgttcactgagcacctgctatgtgctggctgtgtaccaggcactttacacgtgccacaggtgtccagtaaatccccacaacaagcttacgaagtaggtgctatttgtcccctttacaggcagaggagttgagtctccaagaagtgaagtgacttgcccagaatccctcagccgggagtggagtagctgggaaaggcgtggtagagaccatggacgtgggagccaggcagcctacagtggcactcactgctgtgtgaccttgggcaagtcactttacctttcagtgccttggtttcctcatctgtaaatggggataataatagttcctagctcctagcattgttgagtgagcacctgcaatgcgctagg

SEQ ID NO: 20 - DNA, 21 bp, Artificial sequence
  Primer
cctctgccttggtatatccca

SEQ ID NO: 21 - DNA, 20 bp, Artificial sequence
  Primer
cctagcgcattgcaggtgct

SEQ ID NO: 22 - DNA, 851 bp, Homo sapiens
cctagcccatgaattctgtctccagaaagttatgttcagactgtggcttataaaaataagtctgtgacttatgtaacattctgcaaacacaacctaacttttgtttgagacacaccaagttctgactgttctttctatcctccaagaagaaagggataggccgggtgtggtggctcacgcctgtaatcccagcactttgggaggccgaggctggcagatcacgatgtcaggagatcgagaccatcctggctaacacggtgaaaccccttctctactaaaattatacaaaaaaaaaaaaaaaattagccaggcttggtggcagacacctgtagtcccagctactcaggaggctgaggcaggagaatggtgtgaacccgggcggtggagcttgcagtgagctgagatcgcgcccctgcactccagcctgggcgacagaacgagactctgtctcaaaaaaaaaaaaacaaaagaagaagaagaaagggataaatagagcattctgcacagaaatgaaaagagccagcaaaaaaagagaaccaaggaaaaaagatgatggcagaaaagacagtataccaacatgaatgtggctcatgtcgggcccttagctcttcaagttcaaacattctatatttatctcttgggtatggctcccttttgtttctcttattccttgaagtctggctgtatgtatctaaccaagtagaaatatcacacatctgccccctctaccatttaagactctttgaaatccacactcaattagcctatttattggaaagttcctatgactagaaaattcctatgactagacagcactttctttggtaaaaagatgcttcctctgagcaacg

SEQ ID NO: 23 - DNA, 22 bp, Artificial sequence
  Primer
cctagcccatgaattctgtctc

SEQ ID NO: 24 - DNA, 21 bp, Artificial sequence
  Primer
cgttgctcagaggaagcatct

SEQ ID NO: 25 - DNA, 269 bp, Homo sapiens
tatgacctcggaggagctgtggctcgaaccagtgttgggctaaaggcggactggcagggggcagggaagctcaaagatctggggtgctgccaggaaaaagcaaattctggaagttaatggttttgagtgatttttaaatccttgctggcggagaggcccgcctctccccggtatcagcgcttcctcattctttgaatccgcggctccgcggtcttcggcgtcagaccagccggaggaagcctgtttgcaatttaagcgggctgtgaacg

SEQ ID NO: 26 - DNA, 22 bp, Artificial sequence
  Primer
tatgacctcggaggagctgtgg

SEQ ID NO: 27 - DNA, 22 bp, Artificial sequence
  Primer
cgttcacagcccgcttaaattg

SEQ ID NO: 28 - DNA, 209 bp, Homo sapiens
aagccgaggcctcataaatgctgcgacgcacgcccagccgcaaacagccggggctccagcgggagaacgataatgcaaagtgctatgttcttggctgttcaacacgactgcagacccatggacaagagcgcaggcagtggccacaagagcgaggagaagcgagaaaagatgaaacggaccctgtgagtatggctttcttccctctcccg

SEQ ID NO: 29 - DNA, 22 bp, Artificial sequence
  Primer
aagccgaggcctcataaatgct

SEQ ID NO: 30 - DNA, 22 bp, Artificial sequence
  Primer
cgggagagggaagaaagccata

SEQ ID NO: 31 - DNA, 296 bp, Homo sapiens
ctggctcaggctttcacacacacacacgcgcacacacacacacacacacacacggacaggcacccccttggtggccttcacagtttcaccttcaggtaaatgggctcatcctttgagccatgaggatgggaagcgaagcaaggaatgaaaaagctagtgtgtttgtgtgtgtgtgtgtgtgtgtgtgtgtgtgtgagcgcgcgcgcgcgcgcgcgtgtgtactcgtgcgtgtgcctgtgtgtgcctgggagtgacctcacagctgccggaacataaagactcacaggtccgcctcc

SEQ ID NO: 32 - DNA, 22 bp, Artificial sequence
  Primer
ctggctcaggctttcacacaca

SEQ ID NO: 33 - DNA, 22 bp, Artificial sequence
  Primer
ggaggcggacctgtgagtcttt

SEQ ID NO: 34 - DNA, 239 bp, Homo sapiens
cgagcccctctagcgatttgtttaggaaaagtgatgacatgaactagtagtggagaatcgcagcgccgctccccgccctggggagggaggggagccccggagagcctgccggtgggagctggaagcaggctcccggctgagcgccccagcccgaaaggcagggtctgggtgcgggaagagggctcggagctgccttcctgctgccttggggccgcccagatgagggaacagcccgattt

SEQ ID NO: 35 - DNA, 22 bp, Artificial sequence
  Primer
cgagcccctctagcgatttgtt

SEQ ID NO: 36 - DNA, 22 bp, Artificial sequence
  Primer
aaatcgggctgttccctcatct

SEQ ID NO: 37 - DNA, 185 bp, Homo sapiens
tcactcccgaaggtgttgcttccagcttttgcctccttaggaggcagggagcgtcagtgtcgggagaccctgagaccggagtaccgagacgtagctggtgatgcccccgcctgccctcatgtgttctcaggttcttcttatttttattcatctctagaacatggacttcccgtgcctctggctag

SEQ ID NO: 38 - DNA, 22 bp, Artificial sequence
  Primer
tcactcccgaaggtgttgcttc

SEQ ID NO: 39 - DNA, 22 bp, Artificial sequence
  Primer
ctagccagaggcacgggaagtc

SEQ ID NO: 40 - DNA, 186 bp, Homo sapiens
aggagcacgcagagtccatgatggctcagaccaagtgagtgagaggcagagcgaggacgcccctctgctctggcgcgcccggactcggactcgcagactcgcgctggctccagtctctccacgattctctctcccagacttttccccggtcttaagagatcctgtgtccagagggggccttaggta

SEQ ID NO: 41 - DNA, 22 bp, Artificial sequence
  Primer
aggagcacgcagagtccatgat

SEQ ID NO: 42 - DNA, 22 bp, Artificial sequence
  Primer
tacctaaggccccctctggaca

SEQ ID NO: 43 - DNA, 159 bp, Homo sapiens
cagtagggccagggaactgtgagattgtgtcttggactgggacagacagccgggctaaccgcgtgagaggggctcccagatgggcacgcgagttcaggctcttccctactggaagcgccgagcggccgcacctcagggtctctcctggagccagcacag

SEQ ID NO: 44 - DNA, 22 bp, Artificial sequence
  Primer
cagtagggccagggaactgtga

SEQ ID NO: 45 - DNA, 22 bp, Artificial sequence
  Primer
ctgtgctggctccaggagagac

SEQ ID NO: 46 - DNA, 223 bp, Homo sapiens
ttggcgctgaaagtagccattccatgtcttctttcccgccccgcctcttgtgctccccaccgctttcgtgatgtccgcagttgcccacctgcctctacaataaaaaacgcatccctcctcctgcagggtccaccgcaccgggaagccctgtctgtatcagttaccaaccacaattgcagtgagtacgaatcgtggctttcccacagtcaggaaaggcaaggga

SEQ ID NO: 47 - DNA, 22 bp, Artificial sequence
  Primer
ttggcgctgaaagtagccattc

SEQ ID NO: 48 - DNA, 22 bp, Artificial sequence
  Primer
tcccttgcctttcctgactgtg

(19) United States
(12) Patent Application Publication
(10) Pub. No.: US 2013/0071836 A9
(43) Pub. Date: Mar. 21, 2013

(54) COLON CANCER BIOMARKER DISCOVERY

(71) Applicants: Sungwhan An, Daejon (KR); Chiwang Yoon, Daejon (KR); Youngho Moon, Daejon (KR); Tae Jeong Oh, Daejon (KR); Dae Kyoung Yoon, Daejon (KR); Myungsoon Kim, Daejon (KR)

(72) Inventors: Sungwhan An, Daejon (KR); Chiwang Yoon, Daejon (KR); Youngho Moon, Daejon (KR); Tae Jeong Oh, Daejon (KR); Dae Kyoung Yoon, Daejon (KR); Myungsoon Kim, Daejon (KR)

(21) Appl. No.: 11/279,894

(22) Filed: Apr. 15, 2006

Related U.S. Application Data
(63) Continuation-in-part of application No. 10/984,481, filed on Nov. 9, 2004, now abandoned.
Application No. 10/984,481 is a continuation-in-part of application No. 10/983,809, filed on Nov. 8, 2004, now Pat. No. 7,611,841.
(60) Provisional application No. 60/594,531, filed on Apr. 15, 2005.

(51) Int. Cl. C12Q 1/68; C07H 21/04

(52) U.S. Cl. 435/6.11; 536/25.3

(57) ABSTRACT

The present application discloses an epigenetic marker for colon cancer.

### CROSS-REFERENCE To RELATED APPLICATIONS

The present patent application claims the benefit of priority to U.S. Provisional Patent Application No. 60/594,531, filed Apr. 15, 2005. The present application also claims the benefit of priority to U.S. patent application Ser. Nos. 10/984,481, filed Nov. 9, 2004, and 10/983,809, filed Nov. 8, 2004, the contents of which are incorporated by reference in their entirety.

### BACKGROUND OF THE INVENTION

1. Field of the Invention

The invention relates to a systematic approach to discovering biomarkers in colon cancer cell conversion. The invention relates to discovering colon cancer biomarkers. The invention further relates to diagnosis and prognosis of colon cancer using the biomarkers. The invention further relates to early detection or diagnosis of colon cancer.

2. General Background and State of the Art

Despite the current developed state of medical science, five-year survival rate of human cancers, particularly solid cancers (cancers other than blood cancer) that account for a large majority of human cancers, are less than 50%. About two-thirds of all cancer patients are detected at a progressed stage, and most of them die within two years after the diagnosis of cancer. Such poor results in cancer diagnosis and therapy are due not only to the problem of therapeutic methods, but also to the fact that it is not easy to diagnose cancer at an early stage or to accurately diagnose progressed cancer or observe it following therapeutic invention.

In current clinical practice, the diagnosis of cancer typically is confirmed by performing tissue biopsy after history taking, physical examination and clinical assessment, followed by radiographic testing and endoscopy if cancer is suspected. However, the diagnosis of cancer by the existing clinical practices is possible only when the number of cancer cells is more than a billion, and the diameter of cancer is more than 1 cm. In this case, the cancer cells already have metastatic ability, and at least half thereof have already metastasized. Meanwhile, tumor markers for monitoring substances that are directly or indirectly produced from cancers, are used in cancer screening, but they cause confusion due to limitations in accuracy, since up to about half thereof appear normal even in the presence of cancer, and they often appear positive even in the absence of cancer. Furthermore, the anticancer agents that are mainly used in cancer therapy have the problem that they show an effect only when the volume of cancer is small.

The reason why the diagnosis and treatment of cancer are difficult is that cancer cells are highly complex and variable. Cancer cells grow excessively and continuously, invading surrounding tissue and metastasize to distal organs leading to death. Despite the attack of an immune mechanism or anticancer therapy, cancer cells survive, continually develop, and cell groups that are most suitable for survival selectively propagate. Cancer cells are living bodies with a high degree of viability, which occur by the mutation of a large number of genes. In order that one cell is converted to a cancer cell and developed to a malignant cancer lump that is detectable in clinics, the mutation of a large number of genes must occur. Thus, in order to diagnose and treat cancer at the root, approaches at a gene level are necessary.

Recently, genetic analysis is actively being attempted to diagnose cancer. The simplest typical method is to detect the presence of ABL:BCR fusion genes (the genetic characteristic of leukemia) in blood by PCR. The method has an accuracy rate of more than 95%, and after the diagnosis and therapy of chronic myelocytic leukemia using this simple and easy genetic analysis, this method is being used for the assessment of the result and follow-up study. However, this method has the deficiency that it can be applied only to some blood cancers.

Recently, genetic testing using a DNA in serum or plasma is actively being attempted. This is a method of detecting a cancer-related gene that is isolated from cancer cells and released into blood and present in the form of a free DNA in serum. It is found that the concentration of DNA in serum is increased by a factor of 5-10 times in actual cancer patients as compared to that of normal persons, and such increased DNA is released mostly from cancer cells. The analysis of cancer-specific gene abnormalities, such as the mutation, deletion and functional loss of oncogenes and tumor-suppressor genes, using such DNAs isolated from cancer cells, allows the diagnosis of cancer. In this effort, there has been an active attempt to diagnose lung cancer, head and neck cancer, breast cancer, colon cancer, and liver cancer by examining the promoter methylation of mutated K-Ras oncogenes, p53 tumor-suppressor genes and p16 genes in serum, and the labeling and instability of microsatellite (Chen, X. Q. et al., Clin. Cancer Res., 5:2297, 1999; Esteller, M. et al., Cancer Res., 59:67, 1999; Sanchez-Cespedes, M. et al., Cancer Res., 60:892, 2000; Sozzi, G. et al., Clin. Cancer Res., 5:2689, 1999).

In samples other than blood, the DNA of cancer cells can also be detected. A method is being attempted in which the presence of cancer cells or oncogenes in sputum or bronchoalveolar lavage of lung cancer patients is detected by a gene or antibody test (Palmisano, W. A. et al., Cancer Res., 60:5954, 2000; Sueoka, E. et al., Cancer Res., 59:1404, 1999). Additionally, other methods of detecting the presence of oncogenes in feces of colon and rectal cancer patients (Ahlquist, D. A. et al., Gastroenterol., 119:1219, 2000) and detecting promoter methylation abnormalities in urine and prostate fluid (Goessl, C. et al., Cancer Res., 60:5941, 2000) are being attempted. However, in order to accurately diagnose cancers that cause a large number of gene abnormalities and show various mutations characteristic of each cancer, a method, by which a large number of genes are simultaneously analyzed in an accurate and automatic manner, is required. However, such a method is not yet established.

Accordingly, methods of diagnosing cancer by the measurement of DNA methylation are being proposed. When the promoter CpG island of a certain gene is hyper-methylated, the expression of such a gene is silenced. This is interpreted to be a main mechanism by which the function of this gene is lost even when there is no mutation in the protein-coding sequence of the gene in a living body. Also, this is analyzed as a factor by which the function of a number of tumor-suppressor genes in human cancer is lost. Thus, detecting the methylation of the promoter CpG island of tumor-suppressor genes is greatly needed for the study of cancer. Recently, an attempt has actively been conducted to determine promoter methylation, by methods such as methylation-specific PCR (hereinafter, referred to as MSP) or automatic DNA sequencing, for diagnosis and screening of cancer.

In the genomic DNA of mammal cells, there is the fifth base in addition to A, C, G and T, namely, 5-methylcytosine, in which a methyl group is attached to the fifth carbon of the cytosine ring (5-mC). 5-mC is always attached only to the C of a CG dinucleotide (5′-mCG-3′), which is frequently marked CpG. The C of CpG is mostly methylated by attachment with a methyl group. The methylation of this CpG inhibits a repetitive sequence in genomes, such as Alu or transposon, from being expressed. Also, this CpG is a site where an epigenetic change in mammalian cells appears most often. The 5-mC of this CpG is naturally deaminated to T, and thus, the CpG in mammal genomes shows only 1% of frequency, which is much lower than a normal frequency (¼×¼=6.25%).

Regions in which CpG are exceptionally integrated are known as CpG islands. The CpG islands refer to sites which are 0.2-3 kb in length, and have a C+G content of more than 50% and a CpG ratio of more than 3.75%. There are about 45,000 CpG islands in the human genome, and they are mostly found in promoter regions regulating the expression of genes. Actually, the CpG islands occur in the promoters of housekeeping genes accounting for about 50% of human genes (Cross, S. H. & Bird, A. P., Curr. Opin. Gene Develop., 5:309, 1995).

In the somatic cells of normal persons, the CpG islands of such housekeeping gene promoter sites are un-methylated, but imprinted genes and the genes on inactivated X chromosomes are methylated such that they are not expressed during development.

During a cancer-causing process, methylation is found in promoter CpG islands, and the restriction on the corresponding gene expression occurs. Particularly, if methylation occurs in the promoter CpG islands of tumor-suppressor genes that regulate cell cycle or apoptosis, restore DNA, are involved in the adhesion of cells and the interaction between cells, and/or suppress cell invasion and metastasis, such methylation blocks the expression and function of such genes in the same manner as the mutations of a coding sequence, thereby promoting the development and progression of cancer. In addition, partial methylation also occurs in the CpG islands according to aging.

An interesting fact is that, in the case of genes whose mutations are attributed to the development of cancer in congenital cancer but do not occur in acquired cancer, the methylation of promoter CpG islands occurs instead of mutation. Typical examples include the promoter methylation of genes, such as acquired renal cancer VHL (von Hippel Lindau), breast cancer BRCA1, colon cancer MLH1, and stomach cancer E-CAD. In addition, in about half of all cancers, the promoter methylation of p16 or the mutation of Rb occurs, and the remaining cancers show the mutation of p53 or the promoter methylation of p73, p 14 and the like.

An important fact is that an epigenetic change caused by promoter methylation causes a genetic change (i.e., the mutation of a coding sequence), and the development of cancer is progressed by the combination of such genetic and epigenetic changes. In a MLH1 gene as an example, there is the circumstance in which the function of one allele of the MLH1 gene in colon cancer cells is lost due to its mutation or deletion, and the remaining one allele does not function due to promoter methylation. In addition, if the function of MLH1, which is a DNA restoring gene, is lost due to promoter methylation, the occurrence of mutation in other important genes is facilitated to promote the development of cancer.

Most cancers show three common characteristics with respect to CpG, namely, hypermethylation of the promoter CpG islands of tumor-suppressor genes, hypomethylation of the remaining CpG base sites, and an increase in the activity of methylation enzyme, namely, DNA cytosine methyltransferase (DNMT) (Singal, R. & Ginder, G. D., Blood, 93:4059, 1999; Robertson, K. & Jones, P. A., Carcinogensis, 21:461, 2000; Malik, K. & Brown, K. W., Brit. J. Cancer, 83:1583, 2000).

When promoter CpG islands are methylated, the reason why the expression of the corresponding genes is blocked is not clearly established, but is presumed to be because a methyl CpG-binding protein (MECP) or a methyl CpG-binding domain protein (MBD), and histone deacetylase, bind to methylated cytosine thereby causing a change in the chromatin structure of chromosomes and a change in histone protein.

It is unsettled whether the methylation of promoter CpG islands directly causes the development of cancer or is a secondary change after the development of cancer. However, it is clear that the promoter methylation of tumor-related genes is an important index to cancer, and thus, can be used in many applications, including the diagnosis and early detection of cancer, the prediction of the risk of the development of cancer, the prognosis of cancer, follow-up examination after treatment, and the prediction of a response to anticancer therapy. Recently, an attempt to examine the promoter methylation of tumor-related genes in blood, sputum, saliva, feces or urine and to use the examined results for the diagnosis and treatment of various cancers, has been actively conducted (Esteller, M. et al., Cancer Res., 59:67, 1999; Sanchez-Cespedez, M. et al., Cancer Res., 60:892, 2000; Ahlquist, D. A. et al., Gastroenterol., 119:1219, 2000).

In order to maximize the accuracy of cancer diagnosis using promoter methylation, analyze the development of cancer according to each stage and discriminate a change according to cancer and aging, an examination that can accurately analyze the methylation of all the cytosine bases of promoter CpG islands is required. Currently, a standard method for this examination is a bisulfite genome-sequencing method, in which a sample DNA is treated with sodium bisulfite, and all regions of the CpG islands of a target gene to be examined is amplified by PCR, and then, the base sequence of the amplified regions is analyzed. However, this examination has the problem that there are limitations to the number of genes or samples that can be examined at a given time. Other problems are that automation is difficult, and much time and expense are required.

Conventional methods of CpG detection utilize amplification of regions of genes containing CpG island by methylation specific PCR (MSP) together with a base sequence analysis method (bisulfite genome-sequencing method). Furthermore, there is no method that can analyze various changes of the promoter methylation of many genes at a given time in an accurate, rapid and automated manner, and can be applied to the diagnosis, early diagnosis or assessment of each stage of various cancers in clinical practice.

In the area of screening of new tumor suppressor genes associated with methylation, many studies have been performed. Examples of the existing screening methods include: a method where the genomic DNAs of cancer tissues and normal tissues are restricted with methylation-related restriction enzymes, and many DNA fragments obtained are all cloned, and then DNA fragments that are differentially cleaved in cancer tissues and normal tissues are selected, sequenced and screened (Huang, T. H. et al., Hum. Mol. Genet., 8:459, 1999; Cross, S. H. et al., Nat. Genet., 6:236, 1994). However, such methods have shortcomings in that they require much time, and are not efficient to screen gene candidates and also are difficult to apply in actual clinical practice.

Accordingly, the present invention is directed to screening for methylated promoter markers involved in cell conversion especially cancer cell conversion and treatment of cancer.

### SUMMARY OF THE INVENTION

The present invention is directed to a systematic approach to identifying methylation regulated marker genes in colon cancer cell conversion. In one aspect of the invention, (1) the genomic expression content between a converted and unconverted cell or cell line is compared and a profile of the expressed genes that are more abundant in the unconverted cell or cell line is categorized; (2) a converted cell or cell line is treated with a methylation inhibitor, and genomic expression content between the methylation inhibitor treated converted cell or cell line and untreated converted cell or cell line is compared and a profile of the more abundantly expressed genes in the methylation inhibitor treated converted cell or cell line is categorized; (3) profiles of genes from those obtained in (1) and (2) above are compared and the genes that appear in both groups are considered to be candidate methylation regulated marker genes in converting a cell from the unconverted state to the converted form. Further confirmation may be needed such as by examining the sequence of the gene to determine if there is a CpG sequence present, and by carrying out further biochemical assays to determine whether the genes are actually methylated.

The present invention is also based on the finding that by using this system several genes are identified as being differentially methylated in colon cancer as well as at various dysplasic stages of the tissue in the progression to colon cancer. This discovery is useful for colon cancer screening, risk-assessment, prognosis, disease identification, disease staging and identification of therapeutic targets. The identification of genes that are methylated in colon cancer and its various grades of lesion allows for the development of accurate and effective early diagnostic assays, methylation profiling using multiple genes, and identification of new targets for therapeutic intervention. Further, the methylation data may be combined with other non-methylation related biomarker detection methods to obtain a more accurate diagnostic system for colon cancer.

In one embodiment, the invention provides a method of diagnosing various stages or grades of colon cancer progression comprising determining the state of methylation of one or more nucleic acid biomarkers isolated from the subject as described above. The state of methylation of one or more nucleic acids compared with the state of methylation of one or more nucleic acids from a subject not having the cellular proliferative disorder of colon tissue is indicative of a certain stage of colon disorder in the subject. In one aspect of this embodiment, the state of methylation is hypermethylation.

In one aspect of the invention, nucleic acids are methylated in the regulatory regions. In another aspect, since methylation begins from the outer boundaries of the regulatory region and working inward, detecting methylation at the outer boundaries of the regulatory region allows for early detection of the gene involved in cell conversion.

In one aspect, the invention provides a method of diagnosing a cellular proliferative disorder of colon tissue in a subject by detecting the state of methylation of one or more of the following exemplified nucleic acids: LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1; ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1, or a combination thereof.

Another embodiment of the invention provides a method of determining a predisposition to a cellular proliferative disorder of colon tissue in a subject. The method includes determining the state of methylation of one or more nucleic acids isolated from the subject, wherein the state of methylation of one or more nucleic acids compared with the state of methylation of the nucleic acid from a subject not having a predisposition to the cellular proliferative disorder of colon tissue is indicative of a cell proliferative disorder of colon tissue in the subject. Some of the exemplified nucleic acids can be nucleic acids encoding LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1; ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1, or a combination thereof.

In yet another embodiment, the invention is directed to early detection of the probable likelihood of formation of colon cancer. According to an embodiment of the instant invention, when a clinically or morphologically normal appearing tissue contains methylated genes that are known to be methylated in cancerous tissue, this is indication that the normal appearing tissue is progressing to cancerous form. Thus, a positive detection of methylation of colon cancer specific genes as described in the instant application in normal appearing colon tissue constitutes early detection of colon cancer.

Still another embodiment of the invention provides a method for detecting a cellular proliferative disorder of colon tissue in a subject. The method includes contacting a specimen containing at least one nucleic acid from the subject with an agent that provides a determination of the methylation state of at least one nucleic acid. The method further includes identifying the methylation states of at least one region of at least one nucleic acid, wherein the methylation state of the nucleic acid is different from the methylation state of the same region of nucleic acid in a subject not having the cellular proliferative disorder of colon tissue.

Yet a further embodiment of the invention provides a kit useful for the detection of a cellular proliferative disorder in a subject comprising carrier means compartmentalized to receive a sample therein; and one or more containers comprising a first container containing a reagent that sensitively cleaves unmethylated nucleic acid and a second container containing target-specific primers for amplification of the biomarker.

In one embodiment, the invention is directed to a method for discovering a methylation marker gene for the conversion of a normal cell to colon cancer cell comprising: (i) comparing converted and unconverted cell gene expression content to identify a gene that is present in greater abundance in the unconverted cell; (ii) treating a converted cell with a demethylating agent and comparing its gene expression content with gene expression content of an untreated converted cell to identify a gene that is present in greater abundance in the cell treated with the demethylating agent; and (iii) identifying a gene that is common to the identified genes in steps (i) and (ii), wherein the common identified gene is the methylation marker gene. This method may further comprise reviewing the sequence of the identified gene and discarding the gene for which the promoter sequence does not have a CpG island. The comparing may be carried out by direct comparison or indirect comparison. The demethylating agent may be 5 aza 2′-deoxycytidine (DAC). In this method, confirming the methylation marker gene may comprise assaying for methylation of the common identified gene in the converted cell, wherein the presence of methylation in the promoter region of the common identified gene confirms that the identified gene is a marker gene.

In another embodiment, in the method according to above, the assay for methylation of the identified gene may be carried out by: (i) identifying primers that span a methylation site within the nucleic acid region to be amplified; (ii) treating the genome of the converted cell with a methylation specific restriction endonuclease; and (iii) amplifying the nucleic acid by contacting the genomic nucleic acid with the primers, wherein successful amplification indicates that the identified gene is methylated, and unsuccessful amplification indicates that the identified gene is not methylated. The converted cell genome may be treated with an isoschizomer of the methylation sensitive restriction endonuclease that cleaves both methylated and unmethylated CpG-sites as a control. Detecting the presence of amplified nucleic acid may be carried out by hybridization with a probe. Further, the probe may be immobilized on a solid substrate. Still further, the amplification may be carried out by PCR, real time PCR, or amplification or linear amplification using isothermal enzyme. Detection of methylation on the outer part of the promoter is indicative of early detection of cell conversion.

In another embodiment, the invention is directed to a method of identifying a converted colon cancer cell comprising assaying for the methylation of the marker gene.

In yet another embodiment, the invention is directed to a method of diagnosing colon cancer or a stage in the progression of the cancer in a subject comprising assaying for the methylation of the marker gene.

In another embodiment, the invention is directed to a method of diagnosing likelihood of developing colon cancer comprising assaying for methylation of a colon cancer specific marker gene in normal appearing bodily sample. The bodily sample may be solid or liquid tissue, stool, serum or plasma.

In yet another embodiment, the invention is directed to a method assessing the likelihood of developing colon cancer by reviewing a panel of colon-cancer specific methylated genes for their level of methylation and assigning level of likelihood of developing colon cancer.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows a schematic diagram for systematic biomarker discovery for colon cancer.

FIG. 2 shows a schematic diagram for a systematic method for discovering colon cancer biomarker. Gene expression level was compared between tumor and paired tumor-adjacent tissue by direct and indirect comparison methods and down regulated genes in tumor cells were obtained from each comparison.

FIG. 3 shows a flowchart for colon cancer biomarker discovery.

FIG. 4 shows a schematic diagram to conduct methylation assay by enzyme digestion and subsequent gene amplification analysis to determine whether a candidate marker gene is actually methylated.

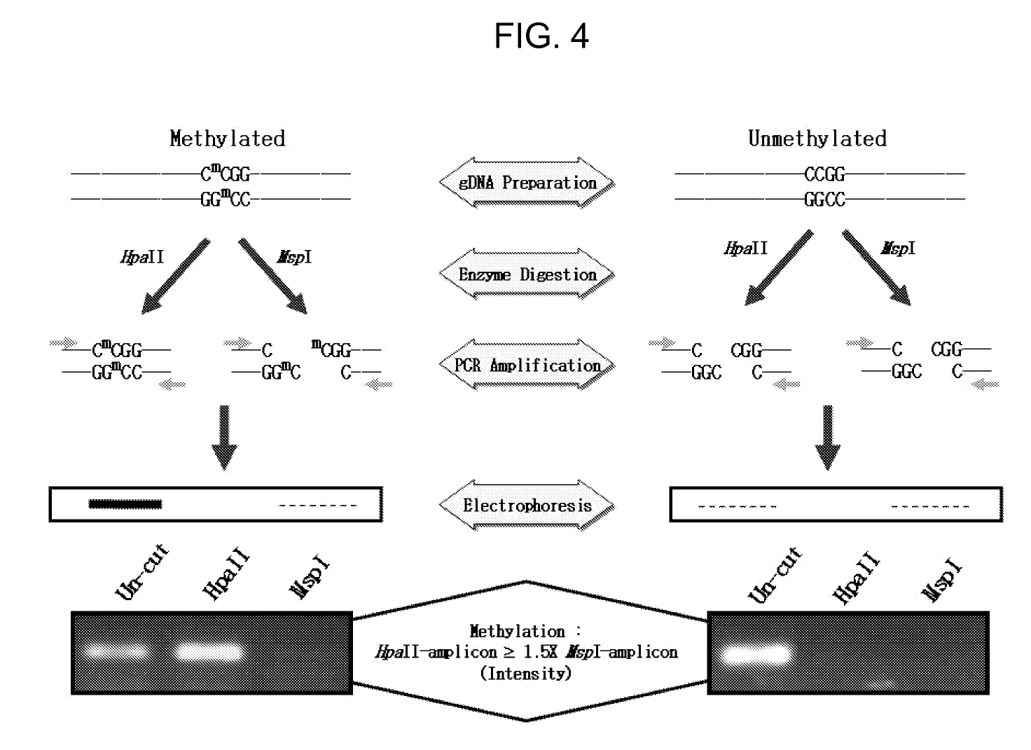

FIGS. 5A and 5B show gene methylation status of 8 identified colon cancer marker genes. FIG. 5A shows methylation assay results of the identified genes by PCR and digestion data of the nucleic acid amplified region with methylation sensitive enzyme in Caco2 cells. FIG. 5B depicts methylation positive genes in Caco2 and HCT116 cells. Black pixels: methylated.

FIG. 6 shows gene expression profile of the 8 identified promoter methylated genes in tumorous and tumor-adjacent non-tumorous colon tissue. These genes were identified based on the genes that were down regulated in colon tumor cells.

FIGS. 7A and 7B show gene methylation status of 8 identified genes in colon cancer. FIG. 7A shows gene methylation status of 8 identified genes in normal tissue from non-patients, and clinical samples from colon tumor and paired tumor-adjacent tissue. FIG. 7B shows methylation frequency of 8 identified markers in normal tissues from non-patients (3 samples), tumor tissues (10 samples) and paired tumor-adjacent tissues (10 samples). The data show that these 8 markers are useful for early detection of colon cancer because they are highly methylated in the paired tumor-adjacent tissues in addition to tumor tissues.

FIG. 8 shows a schematic diagram for a systematic method for discovering additional colon cancer biomarker. Gene expression level was compared between tumor and paired tumor-adjacent tissue cells by indirect comparison method and down regulated genes in tumor cells were obtained from the comparison.

FIG. 9 shows a flowchart for additional colon cancer biomarker discovery.

FIG. 10 shows additional methylation positive genes in Caco2 and HCT116 cells. Black pixels: methylated.

FIG. 11 shows gene expression profile of additional 8 identified promoter methylated genes in tumorous and paired tumor-adjacent colon tissue. These genes were identified based on the genes that were down regulated in colon tumor cells.

FIG. 12 shows reactivation of additional 8 colon cancer biomarkers after demethylating agent treatment.

FIG. 13 shows gene methylation status of 8 identified genes in normal tissue from non-patients, and clinical samples from colon tumor and paired tumor-adjacent tissue.

FIG. 14 shows methylation frequency of 8 identified markers in normal tissue from non-patients (3 samples), tumor tissues (10 samples) and paired tumor-adjacent tissues (10 samples). The data show that these 8 markers are useful for early detection of colon cancer because they are highly methylated in the paired tumor-adjacent tissues in addition to tumor tissues.

### DETAILED DESCRIPTION OF THE PREFERRED EMBODIMENTS

In the present application, “a” and “an” are used to refer to both single and a plurality of objects.

As used herein, “cell conversion” refers to the change in characteristics of a cell from one form to another such as from normal to abnormal, non-tumorous to tumorous, undifferentiated to differentiated, stem cell to non-stem cell. Further, the conversion may be recognized by morphology of the cell, phenotype of the cell, biochemical characteristics and so on. There are many examples, but the present application focuses on the presence of abnormal and cancerous cells in the colon. Markers for such tissue conversion are within the purview of colon cancer cell conversion.

As used herein, “demethylating agent” refers to any agent, including but not limited to chemical or enzyme, that either removes a methyl group from the nucleic acid or prevents methylation from occurring. Examples of such demethylating agents include without limitation nucleotide analogs such as 5-azacytidine, 5 aza 2′-deoxycytidine (DAC), arabinofuranosyl-5-azacytosine, 5-fluoro-2′-deoxycytidine, pyrimidone, trifluoromethyldeoxycytidine, pseudoisocytidine, dihydro-5-azacytidine, AdoMet/AdoHcy analogs as competitive inhibitors such as AdoHcy, sinefungin and analogs, 5′deoxy-5′-S-isobutyladenosine (SIBA), 5′-methylthio-5′deoxyadenosine (MTA), drugs influencing the level of AdoMet such as ethionine analogs, methionine, L-cis-AMB, cycloleucine, antifolates, methotrexate, drugs influencing the level of AdoHcy, dc-AdoMet and MTA such as inhibitors of AdoHcy hydrolase, 3-deaza-adenosine, neplanocin A, 3-deazaneplanocin, 4′-thioadenosine, 3-deaza-aristeromycin, inhibitors of ornithine decarboxylase, α-difluoromethylornithine (DFMO), inhibitors of spermine and spermidine synthetase, S-methyl-5′-methylthioadenosine (MTA), L-cis-AMB, AdoDATO, MGBG, inhibitors of methylthioadenosine phosphorylase, difluoromethylthioadenosine (DFMTA), other inhibitors such as methinin, spermine/spermidine, sodium butyrate, procainamide, hydralazine, dimethylsulfoxide, free radical DNA adducts, UV-light, 8-hydroxy guanine, N-methyl-N-nitrosourea, novobiocine, phenobarbital, benzo[a]pyrene, ethylmethansulfonate, ethylnitrosourea, N-ethyl-N′-nitro-N-nitrosoguanidine, 9-aminoacridine, nitrogen mustard, N-methyl-N′-nitro-N-nitrosoguanidine, diethylnitrosamine, chlordane, N-acetoxy-N-2-acetylaminofluorene, aflatoxin B1, nalidixic acid, N-2-fluorenylacetamine, 3-methyl-4′-(dimethylamino)azobenzene, 1,3-bis(2-chlorethyl)-1-nitrosourea, cyclophosphamide, 6-mercaptopurine, 4-nitroquinoline-1-oxide, N-nitrosodiethylamine, hexamethylenebisacetamide, retinoic acid, retinoic acid with cAMP, aromatic hydrocarbon carcinogens, dibutyryl cAMP, or antisense mRNA to the methyltransferase (Zingg et al., Carcinogenesis, 18:5, pp. 869-882, 1997). The contents of this reference is incorporated by reference in its entirety especially with regard to the discussion of methylation of the genome and inhibitors thereof.

As used herein, “direct comparison” refers to a competitive binding to a probe among differentially labeled nucleic acids from more than one source in order to determine the relative abundance of one type of differentially labeled nucleic acid over the other.

As used herein, “early detection” of cancer refers to the discovery of a potential for cancer prior to metastasis, and preferably before morphological change in the subject tissue or cells is observed. Further, “early detection” of cell conversion refers to the high probability of a cell to undergo transformation in its early stages before the cell is morphologically designated as being transformed.

As used herein, “hypermethylation” refers to the methylation of a CpG island.

As used herein, “indirect comparison” refers to assessing the level of nucleic acid from a first source with the level of the same allelelic nucleic acid from a second source by utilizing a reference probe to which is separately hybridized the nucleic acid from the first and second sources and the results are compared to determine the relative amounts of the nucleic acids present in the sample without direct competitive binding to the reference probe.

As used herein, “sample” or “bodily sample” is referred to in its broadest sense, and includes any biological sample obtained from an individual, body fluid, cell line, tissue culture, depending on the type of assay that is to be performed. As indicated, biological samples include body fluids, such as semen, lymph, sera, plasma, stool, and so on. Methods for obtaining tissue biopsies and body fluids from mammals are well known in the art. A tissue biopsy of the colon is a preferred source.

As used herein, “tumor-adjacent tissue” or “paired tumor-adjacent tissues” refers to clinically and morphologically designated normal appearing tissue adjacent to the cancerous tissue region.

Screening for Methylation Regulated Biomarkers

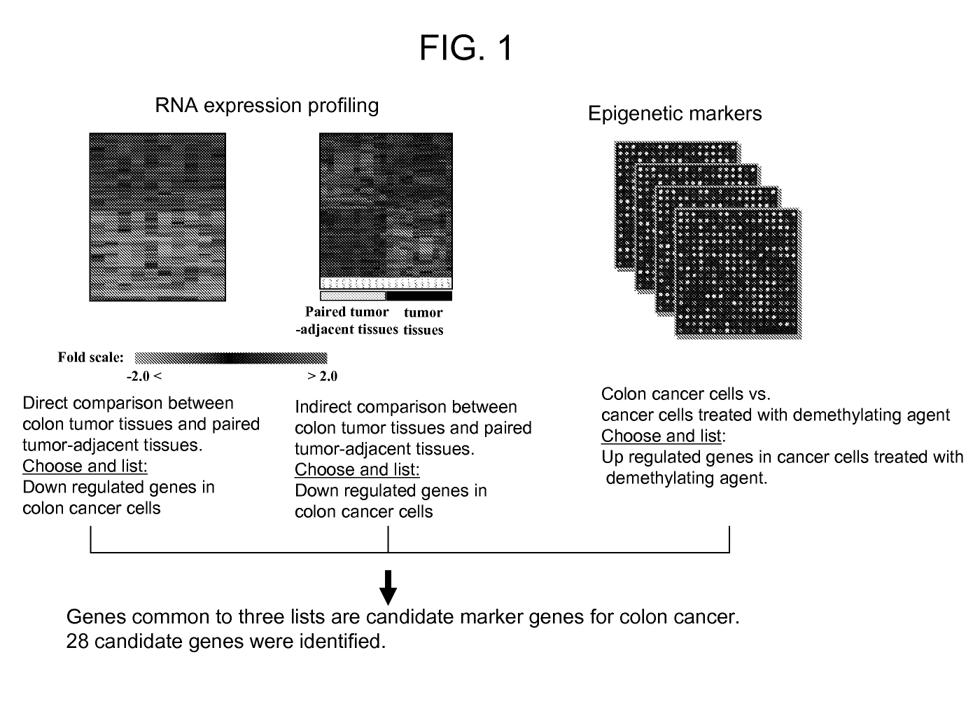

The present invention is directed to a method of determining biomarker genes that are methylated when the cell or tissue is converted or changed from one type of cell to another. As used herein, “converted” cell refers to the change in characteristics of a cell or tissue from one form to another such as from normal to abnormal, non-tumorous to tumorous, undifferentiated to differentiated and so on. See FIG. 1.

Thus, the present invention is directed to a systematic approach to identifying methylation regulated marker genes in colon cancer cell conversion. In one aspect of the invention, (1) the genomic expression content between a converted colon cancer and unconverted cell or cell line is compared and a profile of the more abundantly expressed genes in the unconverted cell or cell line is categorized; (2) a converted colon cancer cell or cell line is treated with a methylation inhibitor, and genomic expression content between the methylation inhibitor treated converted colon cancer cell or cell line and untreated converted colon cancer cell or cell line is compared and a profile of the more abundantly expressed genes in the methylation inhibitor treated converted colon cancer cell or cell line is categorized; (3) profiles of genes from those obtained in (1) and (2) above are compared and overlapping genes are considered to be methylation regulated marker genes in converting a cell from the unconverted state to the converted colon cancer cell form.

In addition to the above, in order to further fine-tune the list of candidate biomarkers and also to determine whether the candidate biomarkers so obtained above are indeed methylated under conversion conditions, a nucleic acid methylation detecting assay is carried out. Any number of numerous ways of detecting methylation on a DNA fragment may be used. By way of example only and without limitation, one such way is as follows. Genomic DNA is treated with a methylation sensitive restriction enzyme, and probed with marker specific gene sequence directed to the methylation region. Detection of an uncleaved probed region indicates that methylation has occurred at the probed site.

One way to practice the invention is by utilizing microarray technology as follows:

(1) Converted cell expression library and non-converted cell expression library are differentially labeled with preferably fluorescent labels, Cy3 which produces green color, and Cy5 which emanates red color. They are competitively bound to a microarray immobilized with a set of known gene probes. The genes that are differentially more expressed in the unconverted cells are identified. Alternatively, an indirect comparison method may be used.

(2) Converted cell line is treated with a demethylating agent and the expression library is labeled with a fluorescent label. A differentially labeled expression library from a converted cell line that has not been treated with the demethylating agent is also obtained. The two libraries are competitively bound on a microarray substrate immobilized with a set of known gene probes. The genes that are differentially more expressed in the converted cells treated with the demethylating agent are identified. These genes are presumably reactivated under demethylating conditions. Alternatively, an indirect comparison method may be used.

(3) The identified genes from the two sets of experiments above are compared and genes common to both lists are chosen.

Again, it is understood that such comparison in gene expression between the converted and unconverted cells and between cells treated with demethylating agent and not treated with demethylating agent may be carried out by direct competitive binding to a set of probes. Alternatively, the comparison may be indirect. For instance, the expressed genes may be bound to a set of known reference gene probes each separately. Thus, the relative abundance of expressed genes from the various cells can be compared indirectly. The set of reference gene probes are generally optimized so that they contain as complete a set of expressed genes as possible. See FIGS. 1, 2 and 8.

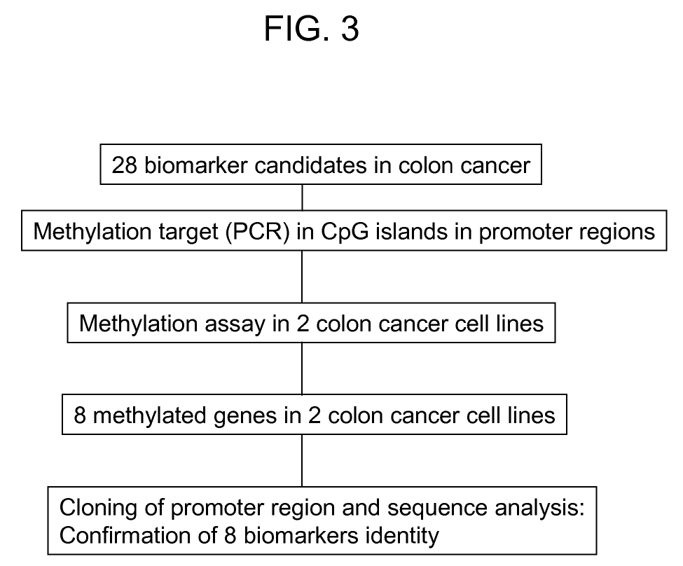

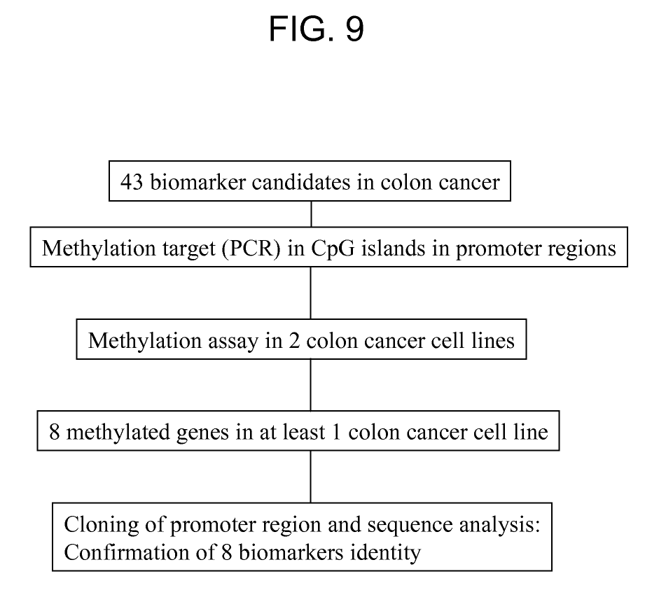

(4) The nucleic acid sequence of the promoter regions of the genes are examined to determine whether there are CpG islands within them. Genes with promoters that do not possess CpG islands are discarded. The remaining genes are assayed for their level of methylation. This can be accomplished using a variety of means. In one embodiment, the genome from converted cells is digested with methylation sensitive restriction endonuclease. Nucleic acid amplification is carried out using various primers wherein the methylation site is located within the region to be amplified. When the nucleic acid amplification step is carried out, successful amplification indicates that methylation has occurred because the gene was not cleaved by the methylation sensitive restriction endonuclease. The absence of an amplified product indicates that methylation did not occur because the gene was digested by the methylation sensitive restriction endonuclease. Results of such experiments are shown in FIGS. 3 and 9.

Colon Cancer Biomarkers

Biomarkers for colon cancer detection is provided in the present application.

Colon Cancer Biomarker—Using Cancer Tumor Cells for Comparison with Normal Cells

In practicing the invention, it is understood that “normal” cells are those that do not show any abnormal morphological or cytological changes. “Tumor” cells are cancer cells. “Non-tumor” cells are those cells that were part of the diseased tissue but were not considered to be the tumor portion.

Colon tumor cell gene expression content was indirectly compared between non-tumor cell and tumor cell gene expression content in a microarray competitive hybridization format. A common reference was competed with non-tumor tissue, such as tumor-adjacent tissue, gene content; and common reference was also competed with tumor cell gene content. Genes that were repressed in tumor cells as compared with non-tumor cells were found and noted as the tumor suppressed genes.

Alternatively, the gene expression content from tumor may be directly competed with non-tumor and/or normal cells in a microarray hybridization format to obtain the tumor suppressed genes. Also, both direct and indirect methods may be used to obtain the tumor suppressed genes.

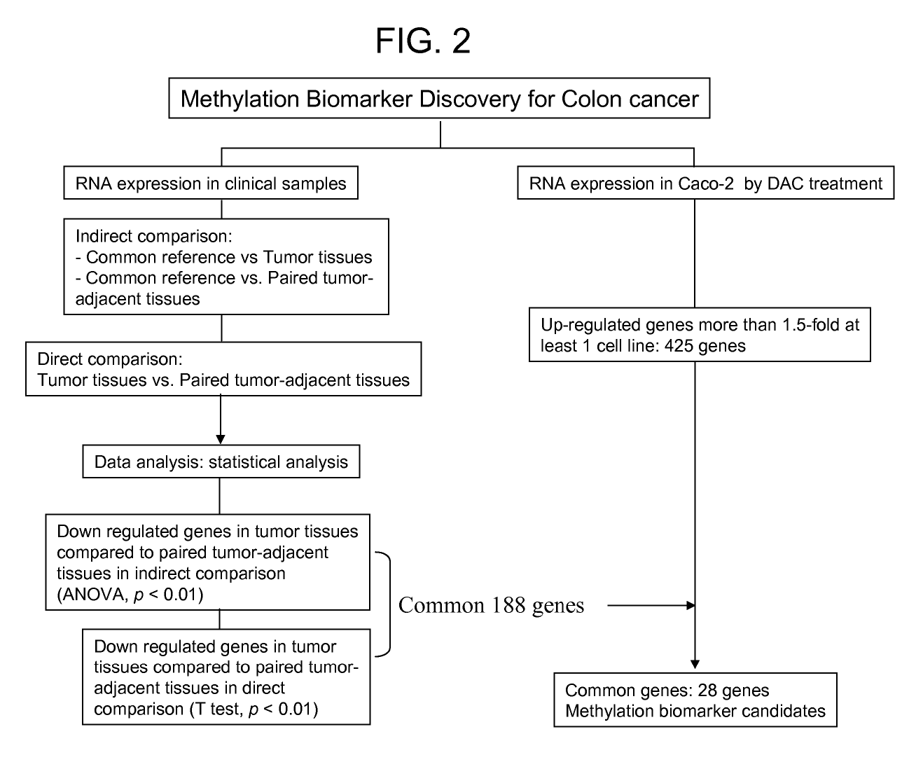

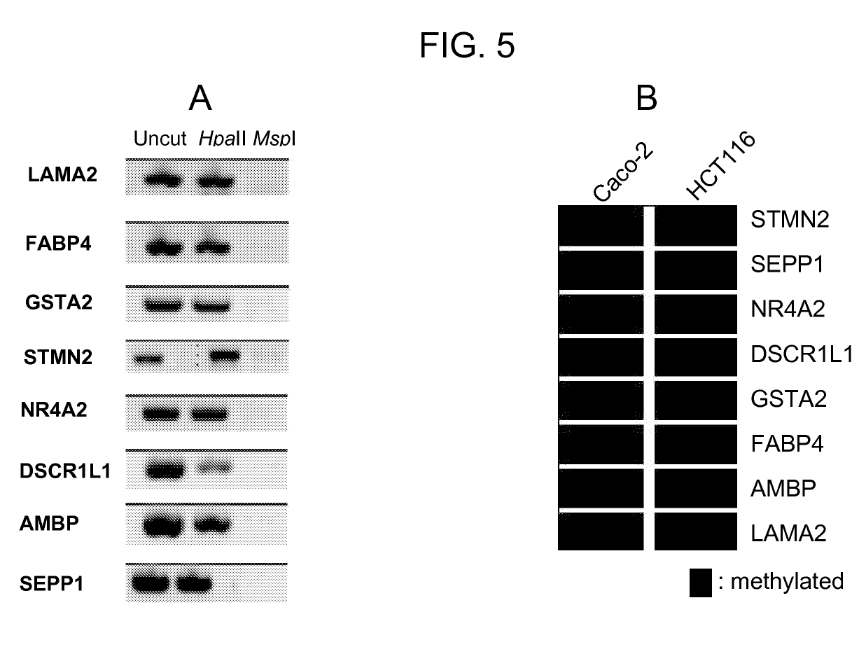

Separately, a colon cancer cell line Caco-2 was treated with a demethylating agent DAC and assayed for reactivation of genes that are normally repressed in tumor cells. Overlapping genes between the tumor suppressed gene set and the demethylation reactivated gene set were considered to be candidate genes for colon cancer biomarkers. Twenty eight (28) such overlapping genes were found (FIG. 2). These genes were then analyzed in silico to determine whether they contained the requisite CpG island motif. A few genes (6 genes) did not contain them and were removed. Further biochemical testing of the remaining 22 genes was needed to determine whether the candidate genes were actually methylated when isolated from tumor cells. Methylation sensitive enzyme/nucleic acid sequence based amplification analysis such as Hpa II/MspI enzyme digestion/PCR (or enzyme digestion post-PCR) further removed a few other genes (14 genes) that were not methylated in any of the two colon cancer cell lines (Caco-2 and HCT116). See FIGS. 5A and 5B. To further confirm biochemically that the candidate gene was indeed methylated in tumor cells, bisulfite sequencing assays were conducted and methylation of the final 8 genes was verified.

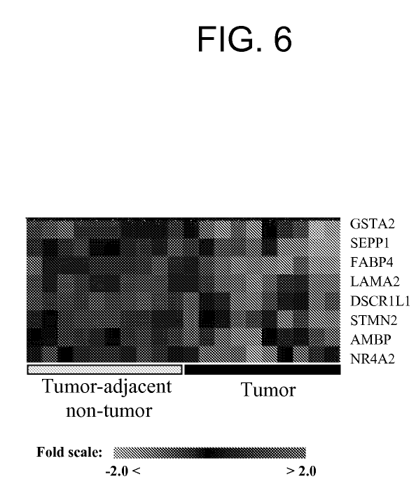

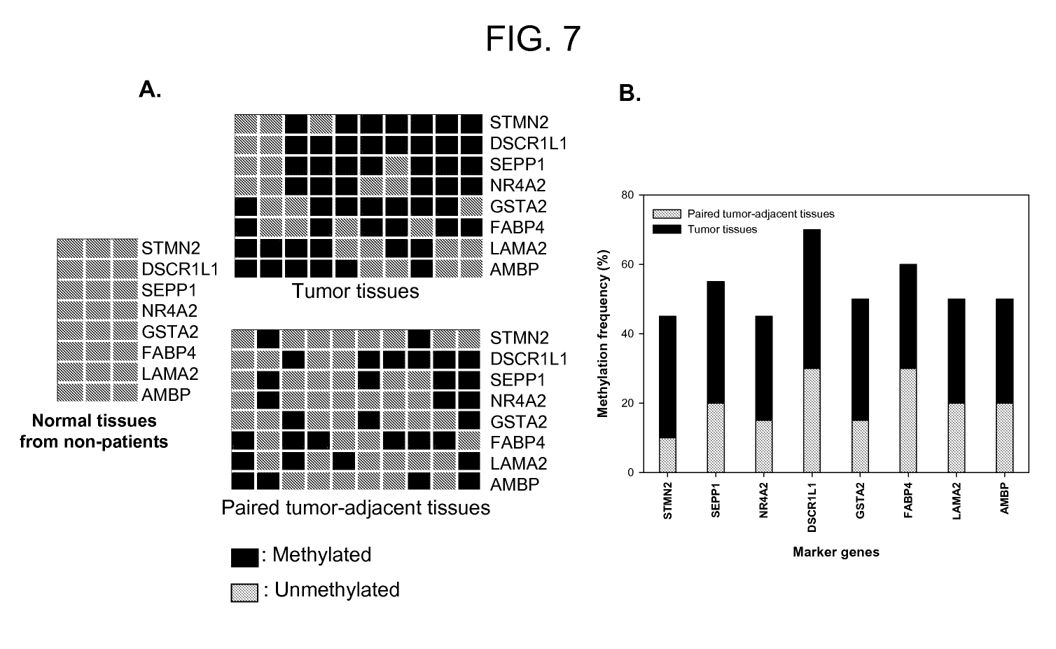

Gene expression profiles of the 8 genes were created. The expression level of the 8 genes was measured in tumor and tumor-adjacent non-tumor tissue (FIG. 6). Methylation status of the genes was also measured using methylation sensitive enzyme/nucleic acid sequence based amplification analysis such as Hpa II/MspI enzyme digestion/PCR (or enzyme digestion post-PCR) method on clinical samples and the results for the 8 genes is shown in FIG. 7. The identified genes are not methylated in normal cells. However, they are methylated in tumor cells as well as in tumor-adjacent non-tumor cells. FIG. 7B shows that the frequency of methylation in tumor cells is higher than in tumor-adjacent tissue.

Thus, one aspect of the invention is in part based upon the discovery of the relationship between colon cancer and the above 8 exemplified promoter hypermethylation of the following genes: LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1 or a combination thereof.

Using the above described method, additional marker genes were also identified as is described infra. They include:

ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1.

In another aspect, the invention provides early detection of a cellular proliferative disorder of colon tissue in a subject comprising determining the state of methylation of one or more nucleic acids isolated from the subject, wherein the state of methylation of one or more nucleic acids as compared with the state of methylation of one or more nucleic acids from a subject not having the cellular proliferative disorder of colon tissue is indicative of a cellular proliferative disorder of colon tissue in the subject. A preferred nucleic acid is a CpG-containing nucleic acid, such as a CpG island.

Another embodiment of the invention provides a method of determining a predisposition to a cellular proliferative disorder of colon tissue in a subject comprising determining the state of methylation of one or more nucleic acids isolated from the subject, wherein the nucleic acid may encode LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1; ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1, and combinations thereof; and wherein the state of methylation of one or more nucleic acids as compared with the state of methylation of said nucleic acid from a subject not having a predisposition to the cellular proliferative disorder of colon tissue is indicative of a cell proliferative disorder of colon tissue in the subject.

As used herein, “predisposition” refers to an increased likelihood that an individual will have a disorder. Although a subject with a predisposition does not yet have the disorder, there exists an increased propensity to the disease.

Another embodiment of the invention provides a method for diagnosing a cellular proliferative disorder of colon tissue in a subject comprising contacting a nucleic acid-containing specimen from the subject with an agent that provides a determination of the methylation state of nucleic acids in the specimen, and identifying the methylation state of at least one region of at least one nucleic acid, wherein the methylation state of at least one region of at least one nucleic acid that is different from the methylation state of the same region of the same nucleic acid in a subject not having the cellular proliferative disorder is indicative of a cellular proliferative disorder of colon tissue in the subject.

The inventive method includes determining the state of methylation of one or more nucleic acids isolated from the subject. The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The nucleic acid of interest can be any nucleic acid where it is desirable to detect the presence of a differentially methylated CpG island. The CpG island is a CpG rich region of a nucleic acid sequence. The nucleic acids includes, for example, a sequence encoding the following genes (GenBank Accession Numbers are shown):

1. LAMA2 (NT_025741); laminin alpha2 (merosin,
congenital)

| | |
|---|---|
| Amplicon size: 1004 bp | |
| ccagtg gcccattcag aagtctaagg acaaaat | (SEQ ID NO:1) |
| atg ttgtaggact gtcctgacca ctgagggaca | |
| gctcacaccc tgacccaaca gtacactaat | |
| gcctgcagta cccaccgttc ccggagtaaa | |
| ccaaaagaca tcctgcgact cacaaaatcc | |
| attatggagt tgttttaaac cttatgagaa | |
| ccactgacct aggccataaa aaacaaatga | |
| aaaaacaaca aaaactagga atggcagtag | |
| ttctgttgag ttaaagagga gaaagaggag | |
| aaaccaggac tggaagagat gaaattgtga | |
| gtctggaagg aaactattgc aaaggccttt | |
| attaccttta agtaaatgtc tcctaactga | |
| actgaaagcc ttcattctaa ccattagttt | |
| ggtcaggaac atttcaggga cgccagggtg | |
| gctgttttat tttgcacttc ctttactgcc | |
| ctcccagcag cctacctagc agtaagttcc | |
| ccagcctgca gtgtccccag agggcacctt | |
| ccctcggcta gtaacttctc agacactctg | |
| gccttgggga tgtcttggat cttctaggta | |
| cacagtggct gcacacagtt ttgccaggcc | |
| tttctcaaga ggtaaaagtt cctagctgtt | |
| tgcatttccc agaaataatg ttttccatgt | |
| gtagggattt tacagatttc aaagtgcttt | |
| catgtcaatt actttcttta atttaaaaga | |
| agttcagata ccaggtcaag ctaggaatga | |
| tccggtctca gagggaagga gcgctctagg | |
| aaaggaggat cctttaatag agggccgtcc | |
| tggggccgcg tgcccatgga aggcgagagt | |
| ggaggagtgt cctctttctc ccccaccctc | |
| aggcggcggc ccggccaaag ccagaggggg | |
| ctgtctcctc ctcttcccca gcagctgctg | |
| ctcgctcagc tcacaagcca aggccagggg | |
| acagggcggc agcgactcct ctggctcccg | |
| agaagtgg | |
| LAMA2-F: | |
| 5′-cca gtg gcc cat tca gaa gtc-3′ | (SEQ ID NO:2) |
| LAMA2-R: | |
| 5′-cca ctt ctc ggg agc cag ag-3′ | (SEQ ID NO:3) |

2. FABP4 (NT_008183); Adipocyte acid binding
protein 4

| | |
|---|---|
| Amplicon size: 1,018 bp | |
| ggatacaca gtgtagcgat gcatcactct | (SEQ ID NO:4) |
| gaaatatttt agtttctttt tttcccctaa | |
| atctgggtat gttcgtggga atttgcagca | |
| catgtgaaca acttctgtca ttcttgcatg | |
| aggcaaaggg aattgaaaac cacgattact | |
| ttagaaaact agtttcacag attggtcact | |
| gtataaaaga aggatattgg ttttggtagc | |
| ttgtgaccac acaccatttc tgatctgaat | |
| aaattcagaa cttataatac agttcagaaa | |
| ttgaatgcag tttctcaata tgaggaaagt | |
| attttagaat aaggcctatt tttcaaagga | |
| tctgtggaaa tcaatgctat gctctcattt | |
| aggagatgga aagagtgagg ttaaattatc | |
| atttcgatta aatctacagt ccagattact | |
| ggtggatgaa ttgaatgtac ttttttattc | |
| atataaaaca tttgaaatca gaaatctgga | |
| gtacttttaa atcccattat ttattttgtt | |
| ttaatcgcca ggtaattcct gagacaggag | |
| tgtcccgaag agcctttgca attatgtaag | |
| aatctccgag gcagttctta tgttcctcaa | |
| ttcaaaagaa ccacataact gcaatttaaa | |
| taacacccca cacacacaca aaataaggtc | |
| gaagtttatc tcaaaataat ttcccctctc | |
| tacactggga taaatatgta taggaataat | |
| agggggaaat tcagtgcact gagcattaag | |
| ctgtcaaaac aggaatgttt aaaatatcct | |
| gttagtggtt taaaaataat ttgtactcta | |
| agtccagtga ctatttgcca gggagaacca | |
| aagttgagaa atttctatta aaaacatgac | |
| tcagagaaaa aaatgcagag gccggtaatg | |
| aaggaaatga ttggatctca ttcccaattg | |
| gtcattccta agatcacatg ttctgagcat | |
| ctttaaaagg aagttatctg gactcaagag | |
| ggtcacagca ccctcctgaa aactgcagc | |
| FABP4-F: | |
| 5′-gga tac aca gtg tag cga tgc a-3′ | (SEQ ID NO:5) |
| FABP4-R: | |
| 5′-gct gca gtt ttc agg agg gtg-3′ | (SEQ ID NO:6) |

3. GSTA2 (NT_007592); glutathione S transferase A2

| | |
|---|---|
| Amplicon size: 1,046 bp | |
| ggtagcag tctcctggag gtttctctaa | (SEQ ID NO:7) |
| gcctgagtga atgaatgaat gaatgaatga | |
| ataattgaaa cgatagaatc aaaaatgtac | |
| tttaggatgt atggttgaaa accacaaaca | |
| atgctgaaga agaacctgcc ttcttcatga | |
| cggtgttgga ggagttcccg gaatgttttc | |
| ttggctcaaa ttattaccca gcagtggcca | |
| ccctcagatt ccagcaaacc agtctcaagt | |
| tttcactgtt taactctgaa ttttcttggc | |
| agcctaagag gtgagagtat gtggtaataa | |
| tacatgtata ggagttaatg ggaagaggaa | |
| gaattcaaga actaatattt actgaaaact | |
| tcctagtgat ccttcctcaa tgctagtccc | |
| tttcaatatt ttatatcttt aaccctcctt | |
| atagtcccat gaaatgatta tctccatttt | |
| ttcgttaatg aaatggaaca tcagagaaat | |
| acaatgttca cagtcacact ccggttggtg | |
| atggacatga atatctacac caaggactaa | |
| aatgaaatca tgcctggaag ccagctgggt | |
| gaaggccctg ggaacccatg aactggccat | |
| gaaaccagag gatgtcactg acagggagga | |
| ccggctggga gctaaatcac tcttcagctc | |
| tttggctgtg agactgcatt tgatcaaaac | |
| cagaaattag gcctcagact tgtttaactg | |
| tagctagaag atccaaattc tttcaagaga | |
| cagagattgt ttatcccttg cttcttttgg | |
| aattctgtat tctaactcta tggggtgcat | |
| tttgttttat aagctggaag aagagatgtt | |
| gctgcattaa ttttgcaata tggaaggagc | |
| tagcatttgt tcaacatcag tcacacactg | |
| atcattcttc taaacttctc tttgttttat | |
| cctacaaaaa ttacctaagg ttaatgtggt | |
| tgttattctc attttacatt tgaggatact | |
| gaggttttta aagtaacttg ctcagagtaa | |
| atgatagatc tgggatccag ggtcactgc | |
| GSTA2-F: | |
| 5′-ggt agc agt ctc ctg gag gtt-3′ | (SEQ ID NO:8) |
| GSTA2-R: | |
| 5′-gca gtg acc ctg gat ccc ag-3′ | (SEQ ID NO:9) |

4. STMN2 (NT_008183); Stathmin-like 2

| | |
|---|---|
| Amplicon size: 736 bp | |
| c cttcctctgt gccaagggaa gaaaacacca | (SEQ ID NO:10) |
| tgcttccctc tcctgagaga ccttcaagag | |
| tttagagacg cctaagtcca gctgtgctaa | |
| aagataaagg acaataatgc aagtctgctc | |
| tgagctgcca ccatagccag acatagggta | |
| gccctgaaag actagaacca aggacagagc | |
| caaaggtgaa agaaaatatg aaaaagtgaa | |
| aacacagtat ttaaacagaa ttttccaaca | |
| gcctgcatat agtgaggact tctcaagctt | |
| ctgaatcctt ttccattgaa ttgtgcaatg | |
| gcacatgtat gagcaaagtc aagcctcctg | |
| gctcccaggt aggcacagcc cagttcttag | |
| ctcctaggaa gcttcagggc ttaaagctcc | |
| actctacttg gactgtacta tcaggccccc | |
| aaaatggggg gagccgacag ggaaggactg | |
| atttccattt caaactgcat tctggtactt | |
| tgtactccag caccattggc cgatcaatat | |
| ttaatgcttg gagattctga ctctgcggga | |
| gtcatgtcag gggaccttgg gagccaatct | |
| gcttgagctt ctgagtgata attattcatg | |
| ggctcctgcc tcttgctctt tctctagcac | |
| ggtcccactc tgcagactca gtgccttatt | |
| cagtcttctc tctcgctctc tccgctgctg | |
| tagccggacc ctttgccttc gccactgctc | |
| agcgtctgca catcc | |
| STMN2-F: | |
| 5′-cct tcc tct gtg cca agg gaa-3′ | (SEQ ID NO:11) |
| STMN2-R: | |
| 5′-gga tgt gca gac gct gag ca-3′ | (SEQ ID NO:12) |

5. NR4A2 (NT_005403); Nuclear receptor subfamily
4 group A, member 2

| | |
|---|---|
| Amplicon size: 919 bp | |
| g ggtgataaca cactcagcct ggtcaactga | (SEQ ID NO:13) |
| acactttctc ccgggctcgg gtacacctgg | |
| agctacaccg gcagcccgcg gcagtcagag | |
| agcatgtagg ggcgcgagag gaaaggcagg | |
| agagagagaa tgctgcagaa caacagttta | |
| gggcgtggaa agactaacca aataaagacc | |
| cagagctaaa aagctactga gggtctacac | |
| tcctgggtat ttccaaacat ctccctcata | |
| cccccccact catcccactc agatgagcct | |
| cttctctgaa gctcagattc agcagtctct | |
| ttctagaaat gaccatctag aaatgaagtt | |
| gaatttcaca atgagaaagt tgttcccgaa | |
| ggcgatgggt cgggcaaacc ttaagttaca | |
| gggtttgcct tgtcctgttt cttgatgttt | |
| ggggagctct ggagagtaaa ggaaagaaat | |
| gaagttgcac taaccttcag ccgagttaca | |
| ggcgttttcg aggaaattaa aggtggacag | |
| tgtcgtaatt caatgaagga caaagtttcc | |
| aagattttta gaaaagcaat ggggagtcca | |
| gcctgtccaa tctcctccct gaaatacaga | |
| cacaggaagc ttcagggttt cttcccgaca | |
| gagattcagc tgggcatatg tagactcacc | |
| aggcaggccc ttccatgcct tccctgtttg | |
| tctcattgga aatgagtggg aagcctttac | |
| caaacatagc acttaaaatg aatgtataaa | |
| agaaatgact tgaaacaaca gaaaatacca | |
| cccattgcac tgtgtaaatc cttgcagaga | |
| gaagatcctg caagaggaaa gctagtccat | |
| gaactcattt aacatttatg atgtaatgaa | |
| ctggcccctt tgctacagtg taggtggaga | |
| ggtcatttcc atcttagg | |
| NR4A2-F: | |
| 5′-ggg tga taa cac act cag cct-3′ | (SEQ ID NO:14) |
| NR4A2-R: | |
| 5′-cct aag atg gaa atg acc tct c-3′ | (SEQ ID NO:15) |

6. DSCR1L1 (NT_007592); Down syndrome cadidate
region gene 1 like-1

| | |
|---|---|
| Amplicon size: 738 bp | |
| gg caacctcaga gttgggagtg aagggaagag | (SEQ ID NO:16) |
| cttccagatg gatattgctg tggccaggcc | |
| tcggtgctgc cttgctctgg ggacggctgg | |
| aggctcgggc tcctggagcc cgctcccact | |
| gcacgcagcc ctccgcgtct gaggcagcac | |
| agcagagtaa cgaacggccc ggctcgctca | |
| tggcaatgac atcaccacaa agactgacac | |
| aagctgaagc tatttttttt tctccaagcc | |
| ttttatctct aggcagtgca gtggagcaaa | |
| ttgaacatga ttatgtgcta aatctgaact | |
| cagactaaat caattcaagc agcgttagct | |
| aggaactgag tcatagctgt tgttgcagcc | |
| gagtgcttat gtttgcaaaa agcaggaggg | |
| ggtgaatctg acaccagagt ttcttctttg | |
| aggtggggga ggtgtaattc tgcagatgag | |
| cctcctgagg ttaaggtttg acaattttct | |
| gccttcgaga tgagcaggaa gttgaggcat | |
| tttgcaaatt gcttggcttc tgttaattgc | |
| tctgtgccac tcagagcagc cacacatgtt | |
| ctgggcatcc taatgcatcc cgggcatggg | |
| ctgaatagaa atccgttctt ggagtgacta | |
| aagagctggt cgtctgtcat ttagggagca | |
| tggtaagagg agataattag aggtttgtgg | |
| aaattctatt tgaaggctat aagtgcagac | |
| cagtaacgct aagagc | |
| DSCR1L1-F: | |
| 5′-ggc aac ctc aga gtt ggg agt-3′ | (SEQ ID NO:17) |
| DSCR1L1-R: | |
| 5′-gct ctt agc gtt act ggt ctg-3′ | (SEQ ID NO:18) |

7. AMBP (NT_008470)-alpha-1-microglobulin/bikunin
precursor

| | |
|---|---|
| Amplicon size: 959 bp | |
| cctctgcctt ggtatatccc acaggctcgg | (SEQ ID NO:19) |
| tctagcaaca gaagggccac cgcctccctg | |
| caacagggca gctgtgaact gaggctgggg | |
| aaggggcctg tggcttgtag ttgacctcag | |
| tgtttgccct gctcagctgg ggccaattac | |
| agccccaagg acagctccaa tcgatccctg | |
| tagcctggct ggggtcagca gtaccaagag | |
| gccgggatgg ctgcttcaga agaggcattg | |
| gccaagcaca atagggccct ggagcaccag | |
| gattgggctc cgccccccaa aagtccccca | |
| cagagggcat gcgaggatgg ggagcgacct | |
| ggcctttctg ctgagtcatg ccatctggac | |
| ctcacagctc tgtgagccag caggtcaagc | |
| ctgattgggc ccatttctca caggaaaaaa | |
| ctgaggccca aggagaggaa gtgacttgcc | |
| agagacctca gggaagtcta tgggcagagc | |
| caagaccaga acccaggtat cctgtctcca | |
| gagttccttc tccagccccc aggcttgccc | |
| tagcctttgc aaataataga gacattaaca | |
| atgatgactg ttacgagctt ccgttcactg | |
| agcacctgct atgtgctggc tgtgtaccag | |
| gcactttaca cgtgccacag gtgtccagta | |
| aatccccaca acaagcttac gaagtaggtg | |
| ctatttgtcc cctttacagg cagaggagtt | |
| gagtctccaa gaagtgaagt gacttgccca | |
| gaatccctca gccgggagtg gagtagctgg | |
| gaaaggcgtg gtagagacca tggacgtggg | |
| agccaggcag cctacagtgg cactcactgc | |
| tgtgtgacct tgggcaagtc actttacctt | |
| tcagtgcctt ggtttcctca tctgtaaatg | |
| gggataataa tagttcctag ctcctagcat | |
| tgttgagtga gcacctgcaa tgcgctagg | |
| A2M-F: | |
| 5′-cct ctg cct tgg tat atc cca-3′ | (SEQ ID NO:20) |
| A2M-R: | |
| 5′-cct agc gca ttg cag gtg ct-3′ | (SEQ ID NO:21) |

8. SEPP1 (NT_006576): selenoprotein P, plasma 1

| | |
|---|---|
| Amplicon size: 851 bp | |
| cctagccca tgaattctgt ctccagaaag | (SEQ ID NO:22) |
| ttatgttcag actgtggctt ataaaaataa | |
| gtctgtgact tatgtaacat tctgcaaaca | |
| caacctaact tttgtttgag acacaccaag | |
| ttctgactgt tctttctatc ctccaagaag | |
| aaagggatag gccgggtgtg gtggctcacg | |
| cctgtaatcc cagcactttg ggaggccgag | |
| gctggcagat cacgatgtca ggagatcgag | |
| accatcctgg ctaacacggt gaaacccctt | |
| ctctactaaa attatacaaa aaaaaaaaaa | |
| aaattagcca ggcttggtgg cagacacctg | |
| tagtcccagc tactcaggag gctgaggcag | |
| gagaatggtg tgaacccggg cggtggagct | |
| tgcagtgagc tgagatcgcg cccctgcact | |
| ccagcctggg cgacagaacg agactctgtc | |
| tcaaaaaaaa aaaaacaaaa gaagaagaag | |
| aaagggataa atagagcatt ctgcacagaa | |
| atgaaaagag ccagcaaaaa aagagaacca | |
| aggaaaaaag atgatggcag aaaagacagt | |
| ataccaacat gaatgtggct catgtcgggc | |
| ccttagctct tcaagttcaa acattctata | |
| tttatctctt gggtatggct cccttttgtt | |
| tctcttattc cttgaagtct ggctgtatgt | |
| atctaaccaa gtagaaatat cacacatctg | |
| ccccctctac catttaagac tctttgaaat | |
| ccacactcaa ttagcctatt tattggaaag | |
| ttcctatgac tagaaaattc ctatgactag | |
| acagcacttt ctttggtaaa aagatgcttc | |
| ctctgagcaa cg | |
| SEPP1-F: | |
| 5′-cct agc cca tga att ctg tct c-3′ | (SEQ ID NO:23) |
| SEPP1-R: | |
| 5′-cgt tgc tca gag gaa gca tct-3′ | (SEQ ID NO:24) |

9. ID3 (NT_004610), inhibitor of DNA binding 3,
dominant negative helix-loop-helix protein

| | |
|---|---|
| Amplicon size: 269 bp | |
| tatgacctcggaggagctgtggctcgaaccagtgtt | (SEQ ID NO:25) |
| gggctaaaggcggactggcagggggcagggaagctc | |
| aaagatctggggtgctgccaggaaaaagcaaattct | |
| ggaagttaatggttttgagtgatttttaaatccttg | |
| ctggcggagaggcccgcctctccccggtatcagcgc | |
| ttcctcattctttgaatccgcggctccgcggtcttc | |
| ggcgtcagaccagccggaggaagcctgtttgcaatt | |
| taagcgggctgtgaacg | |
| Forward; | |
| 5′-tatgacctcggaggagctgtgg-3′ | (SEQ ID NO:26) |
| Reverse; | |
| 5′-cgttcacagcccgcttaaattg-3′ | (SEQ ID NO:27) |

10. RGS2 (NT_004487), regulator of G-protein
signalling 2, 24kDa

| | |
|---|---|
| Amplicon size: 209 bp | |
| aagccgaggcctcataaatgctgcgacgcacgccca | (SEQ ID NO:28) |
| gccgcaaacagccggggctccagcgggagaacgata | |
| atgcaaagtgctatgttcttggctgttcaacacgac | |
| tgcagacccatggacaagagcgcaggcagtggccac | |
| aagagcgaggagaagcgagaaaagatgaaacggacc | |
| ctgtgagtatggctttcttccctctcccg | |
| Forward; | |
| 5′-aagccgaggcctcataaatgct-3′ | (SEQ ID NO:29) |
| Reverse; | |
| 5′-cgggagagggaagaaagccata-3′ | (SEQ ID NO:30) |

11. WISP2 (NT_011362), WNT1 inducible signaling
pathway protein 2

| | |
|---|---|
| Amplicon size: 296 bp | |
| ctggctcaggctttcacacacacacacgcgcacaca | (SEQ ID NO:31) |
| cacacacacacacacacggacaggcacccccttggt | |
| ggccttcacagtttcaccttcaggtaaatgggctca | |
| tcctttgagccatgaggatgggaagcgaagcaagga | |
| atgaaaaagctagtgtgtttgtgtgtgtgtgtgtgt | |
| gtgtgtgtgtgtgtgagcgcgcgcgcgcgcgcgcgt | |
| gtgtactcgtgcgtgtgcctgtgtgtgcctgggagt | |
| gacctcacagctgccggaacataaagactcacaggt | |
| ccgcctcc | |
| Forward; | |
| 5′-ctggctcaggctttcacacaca-3′ | (SEQ ID NO:32) |
| Reverse | |
| 5′-ggaggcggacctgtgagtcttt-3′ | (SEQ ID NO:33) |

12. MGLL (NT_005612), monoglyceride lipase

| | |
|---|---|
| Amplicon size: 239 bp | |
| cgagcccctctagcgatttgtttaggaaaagtgatg | (SEQ ID NO:34) |
| acatgaactagtagtggagaatcgcagcgccgctcc | |
| ccgccctggggagggaggggagccccggagagcctg | |
| ccggtgggagctggaagcaggctcccggctgagcgc | |
| cccagcccgaaaggcagggtctgggtgcgggaagag | |
| ggctcggagctgccttcctgctgccttggggccgcc | |
| cagatgagggaacagcccgattt | |
| Forward; | |
| 5′-cgagcccctctagcgatttgtt-3′ | (SEQ ID NO:35) |
| Reverse; | |
| 5′-aaatcgggctgttccctcatct-3′ | (SEQ ID NO:36) |

13. CPM (NT_029419), carboxypeptidase M 12q14.3

| | |
|---|---|
| Amplicon size: 185 bp | |
| tcactcccgaaggtgttgcttccagcttttgcctcc | (SEQ ID NO:37) |
| ttaggaggcagggagcgtcagtgtcgggagaccctg | |
| agaccggagtaccgagacgtagctggtgatgccccc | |
| gcctgccctcatgtgttctcaggffcttcttatttt | |
| tattcatctctagaacatggacttcccgtgcctctg | |
| gctag | |
| Forward; | |
| 5′-tcactcccgaaggtgttgcttc-3′ | (SEQ ID NO:38) |
| Reverse; | |
| 5′-ctagccagaggcacgggaagtc-3′ | (SEQ ID NO:39) |

14. GABRA1 (NT_023133), gamma-aminobutyric acid
(GABA) A receptor, alpha 1

| | |
|---|---|
| Amplicon size: 186 bp | |
| aggagcacgcagagtccatgatggctcagaccaagt | (SEQ ID NO:40) |
| gagtgagaggcagagcgaggacgcccctctgctctg | |
| gcgcgcccggactcggactcgcagactcgcgctggc | |
| tccagtctctccacgattctctctcccagacttttc | |
| cccggtcttaagagatcctgtgtccagagggggcct | |
| taggta | |
| Forward; | |
| 5′-aggagcacgcagagtccatgat-3′ | (SEQ ID NO:41) |
| Reverse; | |
| 5′-tacctaaggccccctctggaca-3′ | (SEQ ID NO:42) |

15. CLU (NT_023666), clusterin (complement lysis
inhibitor, SP-40, 40, sulfated glycoprotein 2,
testosterone-repressed prostate message 2,
apolipoprotein J)

| | |
|---|---|
| Amplicon size: 159 bp | |
| cagtagggccagggaactgtgagattgtgtcttgga | (SEQ ID NO:43) |
| ctgggacagacagccgggctaaccgcgtgagagggg | |
| ctcccagatgggcacgcgagttcaggctcttcccta | |
| ctggaagcgccgagcggccgcacctcagggtctctc | |
| ctggagccagcacag | |
| Forward; | |
| 5′-cagtagggccagggaactgtga-3′ | (SEQ ID NO:44) |
| Reverse; | |
| 5′-ctgtgctggctccaggagagac-3′ | (SEQ ID NO:45) |

16. F2RL1 (NT_006713), coagulation factor II
(thrombin) receptor-like 1

| | |
|---|---|
| Amplicon size: 223 bp | |
| ttggcgctgaaagtagccattccatgtcttctttcc | (SEQ ID NO:46) |
| cgccccgcctcttgtgctccccaccgctttcgtgat | |
| gtccgcagttgcccacctgcctctacaataaaaaac | |
| gcatccctcctcctgcagggtccaccgcaccgggaa | |
| gccctgtctgtatcagttaccaaccacaattgcagt | |
| gagtacgaatcgtggctttcccacagtcaggaaagg | |
| caaggga | |
| Forward; | |
| 5′-ttggcgctgaaagtagccattc-3′ | (SEQ ID NO:47) |
| Reverse; | |
| 5′-tcccttgcctttcctgactgtg-3′ | (SEQ ID NO:48) |

The bolded “ccgg” refers to sites of methylation, which are also recognized by a methylation sensitive restriction enzyme HpaII.

Methylation

Any nucleic acid sample, in purified or nonpurified form, can be utilized in accordance with the present invention, provided it contains or is suspected of containing, a nucleic acid sequence containing a target locus (e.g., CpG-containing nucleic acid). One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G*C base pairs. In certain regions, the density of CpG doublets reaches the predicted value; it is increased by ten fold relative to the rest of the genome. CpG islands have an average G*C content of about 60%, compared with the 40% average in bulk DNA. The islands take the form of stretches of DNA typically about one to two kilobases long. There are about 45,000 such islands in the human genome.

In many genes, the CpG islands begin just upstream of a promoter and extend downstream into the transcribed region. Methylation of a CpG island at a promoter usually prevents expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

In general, the CpG-containing nucleic acid is DNA. However, invention methods may employ, for example, samples that contain DNA, or DNA and RNA, including messenger RNA, wherein DNA or RNA may be single stranded or double stranded, or a DNA-RNA hybrid may be included in the sample. A mixture of nucleic acids may also be employed. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. The nucleic acid-containing sample used for determination of the state of methylation of nucleic acids contained in the sample or detection of methylated CpG islands may be extracted by a variety of techniques such as that described by Sambrook, et al. (Molecular Cloning: A Laboratory Manual, Cold Spring Harbor, N.Y., 1989; incorporated in its entirety herein by reference).

A nucleic acid can contain a regulatory region which is a region of DNA that encodes information that directs or controls transcription of the nucleic acid. Regulatory regions include at least one promoter. A “promoter” is a minimal sequence sufficient to direct transcription, to render promoter-dependent gene expression controllable for cell-type specific, tissue-specific, or inducible by external signals or agents. Promoters may be located in the 5′ or 3′ regions of the gene. Promoter regions, in whole or in part, of a number of nucleic acids can be examined for sites of CG-island methylation. Moreover, it is generally recognized that methylation of the target gene promoter proceeds naturally from the outer boundary inward. Therefore, early stage of cell conversion can be detected by assaying for methylation in these outer areas of the promoter region.

Nucleic acids isolated from a subject are obtained in a biological specimen from the subject. If it is desired to detect colon cancer or stages of colon cancer progression, the nucleic acid may be isolated from colon tissue by scraping or taking a biopsy. These specimen may be obtained by various medical procedures known to those of skill in the art.

In one aspect of the invention, the state of methylation in nucleic acids of the sample obtained from a subject is hypermethylation compared with the same regions of the nucleic acid in a subject not having the cellular proliferative disorder of colon tissue. Hypermethylation, as used herein, is the presence of methylated alleles in one or more nucleic acids. Nucleic acids from a subject not having a cellular proliferative disorder of colon tissues contain no detectable methylated alleles when the same nucleic acids are examined.

Samples

The present application describes early detection of colon cancer. Colon cancer specific gene methylation is described. Applicant has shown that colon cancer specific gene methylation also occurs in tissue that are adjacent to the tumor region. Therefore, in a method for early detection of colon cancer, any bodily sample, including liquid or solid tissue may be examined for the presence of methylation of the colon-specific genes. Such samples may include, but not limited to, serum, stool, or plasma.

Individual Genes and Panel

It is understood that the present invention may be practiced using each gene separately as a diagnostic or prognostic marker or a few marker genes combined into a panel display format so that several marker genes may be detected for overall pattern or listing of genes that are methylated to increase reliability and efficiency. Further, any of the genes identified in the present application may be used individually or as a set of genes in any combination with any of the other genes that are recited in the application. For instance, a criteria may be established where if for example 6, 7, 8, 9, 10, 11, 12 and so forth of 16 or so colon-specific genes are methylated, it indicates a certain level of likelihood of developing cancer. Or, genes may be ranked according to their importance and weighted and together with the number of genes that are methylated, a level of likelihood of developing cancer may be assigned. Such algorithms are within the purview of the invention.

Methylation Detection Methods

Detection of Differential Methylation—Methylation Sensitive Restriction Endonuclease

Detection of differential methylation can be accomplished by contacting a nucleic acid sample with a methylation sensitive restriction endonuclease that cleaves only unmethylated CpG sites under conditions and for a time to allow cleavage of unmethylated nucleic acid. In a separate reaction, the sample is further contacted with an isoschizomer of the methylation sensitive restriction endonuclease that cleaves both methylated and unmethylated CpG-sites under conditions and for a time to allow cleavage of methylated nucleic acid. Specific primers are added to the nucleic acid sample under conditions and for a time to allow nucleic acid amplification to occur by conventional methods. The presence of amplified product in the sample digested with methylation sensitive restriction endonuclease but absence of an amplified product in sample digested with an isoschizomer of the methylation sensitive restriction enzyme endonuclease that cleaves both methylated and unmethylated CpG-sites indicates that methylation has occurred at the nucleic acid region being assayed. However, lack of amplified product in the sample digested with methylation sensitive restriction endonuclease together with lack of an amplified product in the sample digested with an isoschizomer of the methylation sensitive restriction enzyme endonuclease that cleaves both methylated and unmethylated CpG-sites indicates that methylation has not occurred at the nucleic acid region being assayed.

As used herein, a “methylation sensitive restriction endonuclease” is a restriction endonuclease that includes CG as part of its recognition site and has altered activity when the C is methylated as compared to when the C is not methylated. Preferably, the methylation sensitive restriction endonuclease has inhibited activity when the C is methylated (e.g., SmaI). Specific non-limiting examples of methylation sensitive restriction endonucleases include SmaI, BssHII, or HpaII, BSTUI, and NotI. Such enzymes can be used alone or in combination. Other methylation sensitive restriction endonucleases will be known to those of skill in the art and include, but are not limited to SacII, and EagI, for example. An “isoschizomer” of a methylation sensitive restriction endonuclease is a restriction endonuclease that recognizes the same recognition site as a methylation sensitive restriction endonuclease but cleaves both methylated and unmethylated CGs, such as for example, MspI. Those of skill in the art can readily determine appropriate conditions for a restriction endonuclease to cleave a nucleic acid (see Sambrook et al., Molecular Cloning: A Laboratory Manual, Cold Spring Harbor Press, 1989).

Primers of the invention are designed to be “substantially” complementary to each strand of the locus to be amplified and include the appropriate G or C nucleotides as discussed above. This means that the primers must be sufficiently complementary to hybridize with their respective strands under conditions that allow the agent for polymerization to perform. Primers of the invention are employed in the amplification process, which is an enzymatic chain reaction that produces exponentially increasing quantities of target locus relative to the number of reaction steps involved (e.g., polymerase chain reaction (PCR)). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

Preferably, the method of amplifying is by PCR, as described herein and as is commonly used by those of ordinary skill in the art. However, alternative methods of amplification have been described and can also be employed such as real time PCR or linear amplification using isothermal enzyme. Multiplex amplification reactions may also be used.

Detection of Differential Methylation—Bisulfite Sequencing Method

Another method for detecting a methylated CpG-containing nucleic acid includes contacting a nucleic acid-containing specimen with an agent that modifies unmethylated cytosine, amplifying the CpG-containing nucleic acid in the specimen by means of CpG-specific oligonucleotide primers, wherein the oligonucleotide primers distinguish between modified methylated and non-methylated nucleic acid and detecting the methylated nucleic acid. The amplification step is optional and although desirable, is not essential. The method relies on the PCR reaction itself to distinguish between modified (e.g., chemically modified) methylated and unmethylated DNA. Such methods are described in U.S. Patent No. 5,786,146, the contents of which are incorporated herein in their entirety especially as they relate to the bisulfite sequencing method for detection of methylated nucleic acid.

Substrates

Once the target nucleic acid region is amplified, the nucleic acid can be hybridized to a known gene probe immobilized on a solid support to detect the presence of the nucleic acid sequence.

As used herein, “substrate,” when used in reference to a substance, structure, surface or material, means a composition comprising a nonbiological, synthetic, nonliving, planar, spherical or flat surface that is not heretofore known to comprise a specific binding, hybridization or catalytic recognition site or a plurality of different recognition sites or a number of different recognition sites which exceeds the number of different molecular species comprising the surface, structure or material. The substrate may include, for example and without limitation, semiconductors, synthetic (organic) metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics.

Several types of membranes are known to one of skill in the art for adhesion of nucleic acid sequences. Specific non-limiting examples of these membranes include nitrocellulose or other membranes used for detection of gene expression such as polyvinylchloride, diazotized paper and other commercially available membranes such as GENESCREEN™, ZETAPROBE™ (Biorad), and NYTRAN™. Beads, glass, wafer and metal substrates are included. Methods for attaching nucleic acids to these objects are well known to one of skill in the art. Alternatively, screening can be done in liquid phase.

Hybridization Conditions

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2× SSC/0.1% SDS at about room temperature (hybridization conditions); 0.2× SSC/0.1% SDS at about room temperature (low stringency conditions); 0.2× SSC/0.1% SDS at about 42.degree. C. (moderate stringency conditions); and 0.1.times.SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

Label

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

Kit

Invention methods are ideally suited for the preparation of a kit. Therefore, in accordance with another embodiment of the present invention, there is provided a kit useful for the detection of a cellular proliferative disorder in a subject. Invention kits include a carrier means compartmentalized to receive a sample therein, one or more containers comprising a first container containing a reagent which sensitively cleaves unmethylated cytosine, a second container containing primers for amplification of a CpG-containing nucleic acid, and a third container containing a means to detect the presence of cleaved or uncleaved nucleic acid. Primers contemplated for use in accordance with the invention include those set forth in SEQ ID NOS:1-24, and any functional combination and fragments thereof. Functional combination or fragment refers to its ability to be used as a primer to detect whether methylation has occurred on the region of the genome sought to be detected.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing methylation sensitive restriction endonuclease. One or more container means can also be included comprising a primer complementary to the locus of interest. In addition, one or more container means can also be included containing an isoschizomer of the methylation sensitive restriction enzyme.

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

### EXAMPLES

### Example 1

### Identification of Genes Repressed in Colon Cancer

To identify genes repressed in colon cancer, microarray hybridization experiments were carried out. Microarray hybridizations were performed according to standard protocol (Schena et al, 1995, Science, 270: 467-470). Total RNA was isolated from non-tumor adjacent to tumor part (10 samples) and tumor part (10 samples) of colon cancer patients. To compare relative difference in gene expression level between non-tumor and tumor tissues indirectly, we prepared common reference RNA (indirect comparison). Total RNA was isolated from 11 human cancer cell lines. Total RNA from cell lines and colon tissues were isolated using Tri Reagent (Sigma, USA) according to manufacturer's instructions. To make common reference RNA, equal amount of total RNA from 11 cancer cell lines was combined. The common reference RNA was used as an internal control. To compare relative difference in gene expression levels in non-tumor and tumor tissues, RNAs isolated from non-tumor and tumor tissues were indirectly compared with common reference RNA. 100 ug of total RNA was labeled with Cy3-dUTP or Cy5-dUTP. The common reference RNA was labeled with Cy3 and RNA from colon tissues was labeled with Cy5, respectively. In addition, gene expression level was directly compared between non-tumor with tumor tissues. RNAs from non-tumor and tumor tissues were directly compared. RNAs from non-tumor and tumor tissues were labeled with Cy3 and Cy5, respectively. Both Cy3- and Cy5-labeled cDNA were purified using PCR purification kit (Qiagen, Germany). The purified cDNA was combined and concentrated at a final volume of 27 ul using Microcon YM-30 (Millipore Corp., USA).

Total 80 ul of hybridization mixture contained: 27 ul labeled cDNA targets, 20 ul of 20× SSC, 8 ul of 1% SDS, 24 ul of formamide (Sigma, USA) and 20 ug of human Cot1 DNA (Invitrogen Corp., USA). The hybridization mixtures were heated at 100° C. for 2 min and immediately hybridized to human 17K cDNA (GenomicTree, Inc) microarrays. The arrays were hybridized at 42° C. for 12-16 h in the humidified HybChamber X (GenomicTree, Inc., Korea). After hybridization, microarray slides were imaged using Axon 4000B scanner (Axon Instruments Inc., USA). The signal and background fluorescence intensities were calculated for each probe spot by averaging the intensities of every pixel inside the target region using GenePix Pro 4.0 software (Axon Instruments Inc., USA). Spots were excluded from analysis due to obvious abnormalities. All data normalization, statistical analysis and cluster analysis were performed using GeneSpring 7.2 (Agilent, USA).

To determine relative difference in gene expression levels between non-tumor and tumor tissues, statistical analysis (ANOVA (p<0.01) for indirect comparison and T test (p<0.01) for direct comparison) was performed. From the results of statistical analysis, a total of 188 common genes were down regulated in tumor compared with non-tumor by direct and indirect comparisons.

### Example 2

### Identification of Methylation Controlled Gene Expression

To determine whether the expression of any of the genes identified in Example 1 is controlled by promoter methylation, colon cancer cell line Caco-2 was treated with demethylation agent, 5-aza-2′deoxycytidine (DAC, Sigma, USA) for three days at a concentration of 200 nM. Cells were harvested and total RNA was isolated from treated and untreated cell lines using Tri reagent. To determine gene expression changes by DAC treatment, transcript level between untreated and treated cell lines was directly compared. From this experiment, 425 genes were identified that show elevated expression when treated with DAC compared with the control group which was not treated with DAC. 28 common genes between the 188 tumor repressed genes and the 425 reactivated genes were identified.

### Example 3

### Confirmation of Methylation of Identified Genes

### Example 3.1

### In Silico Analysis of CpG Island in Promoter Region

The promoter regions of the 28 genes were scanned for the presence of CpG islands using MethPrimer (http://itsa.ucsf.edu/˜urolab/methprimer/index1.html). Six genes did not contain the CpG island and were dropped from the common gene list.

### Example 3.2

### Biochemical Assay for Methylation

To biochemically determine the methylation status of the remaining 22 genes, methylation status of each promoter was detected using the characteristics of restriction endonucleases, HpaII (methylation-sensitive) and MspI (methylation-insensitive) followed by PCR. Both enzymes recognize the same DNA sequence, 5′-CCGG-3′. HpaII is inactive when internal cytosine residue is methylated, whereas MspI is active regardless of methylated or not. In the case that the cytosine residue at the CpG site is unmethylated, both enzymes can digest the target sequence. To determine the methylation status of a specific gene, PCR targets containing one or more HpaII sites from CpG islands in the promoter region were selected. 100 ng of genomic DNA from colon cancer cell lines Caco-2 and HCT116 were digested with 5 U of HpaII and 10 U of MspI, respectively and purified using Qiagen PCR purification kit. Specific primers were used to amplify regions of interest. 5 ng of the purified genomic DNA was amplified by PCR using gene-specific primer sets. DNA from undigested control sample was amplified to determine PCR adequacy. The PCR was performed as follows: 94° C., 1 min; 66° C., 1 min; 72° C., 1 min (30 cycles); and 72° C., 10 min for final extension. Each amplicon was separated on a 2% agarose gel containing ethidium bromide. If the band density of HpaII amplicon is 1.5-fold greater than that of MspI amplicon, the target region was considered to be methylated, while less than 1.5-fold was considered to be unmethylated. From this, it was discovered that 14 genes were not methylated, leaving 8 confirmed candidate genes that fit the criteria of being down regulated in tumor, up regulated under demethylation conditions, contains a CpG island in its promoter and is actually methylated in the cancer cell lines. See FIGS. 5A and 5B.

### Example 3.3

### Bisulfite Sequencing of Methylated Promoter

To further confirm the methylation status of the 8 identified genes, the inventors performed bisulfite sequencing of the individual promoters. Upon treatment of the DNA with bisulfite, unmethylated cytosine is modified to uracil and the methylated cytosine undergoes no change. The inventors performed the bisulfite modification according to Sato, N. et al., Cancer Research, 63:3735, 2003, the contents of which are incorporated by reference herein in its entirety especially regarding the use of bisulfite modification method as applied to detect DNA methylation. The bisulfite treatment was performed on 1 μg of the genomic DNA of the colon cancer cell line Caco-2 and HCT 116 using MSP (Methylation-Specific PCR) bisulfite modification kit (In2Gen, Inc., Seoul, Korea). After amplifying the bisulfite-treated Caco-2 and HCT116 genomic DNA by PCR, the nucleotide sequence of the PCR products was analyzed. The results confirmed that the genes were all methylated.

### Example 4

### Gene Expression Profile of the Identified Genes

FIG. 6 shows the gene expression profiles of the 8 genes that were identified. As shown in FIG. 6, gene expression was repressed in the tumor compared with non-tumor tissues.

### Example 5

### Promoter Methylation Assay on Clinical Samples

To determine the clinical applicability of the methylated promoters of the 8 selected genes of the present invention, methylation assay was performed with colon cancer tissues and paired tumor-adjacent tissue. Methylation assay was performed as described supra using restriction enzyme/PCR. As shown in FIGS. 7A and 7B, none of the genes are methylated in the normal tissue. However, all of the genes are methylated in colon cancer tumors. Further, all of the genes are methylated in paired tumor-adjacent tissue as well. As FIG. 7B shows, the methylation frequency in tumor tissue is higher than in paired tumor adjacent tissue.

The relative methylation frequency was calculated as follows: The total number of samples including tumor and paired tumor-adjacent tissues was divided by the number of methylated samples of each gene.

### Example 6

### Additional Identification of Genes Repressed in Colon Cancer

To identify other genes repressed in colon cancer, microarray hybridization experiments were carried out, as set forth in Example 1 above. Total RNA was isolated from tumor-adjacent tissue (5 samples) and tumor tissue (5 samples) of colon cancer patients. Indirect comparison was carried out between these two types of tissue. The rest of the experimental protocol was carried out as described in Example 1 above.

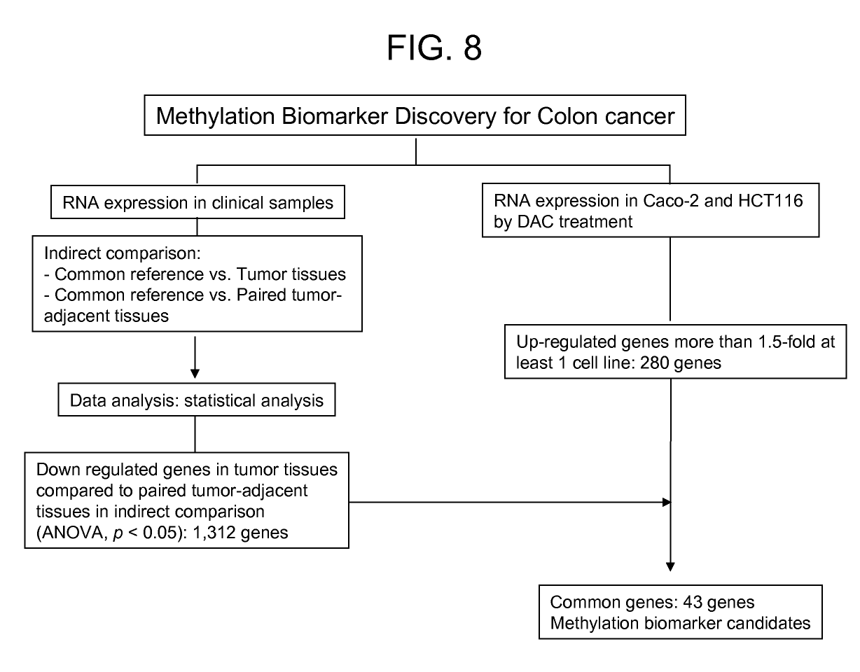

To determine relative difference in gene expression levels between non-tumor and tumor tissues, statistical analysis (ANOVA (p<0.05) for indirect comparison was performed. From the results of the statistical analysis, a total of 1312 genes were down regulated in tumor compared with non-tumor by indirect comparison (FIG. 8).

### Example 7

### Identification of Methylation Controlled Gene Expression

To determine whether the expression of any of the genes identified in Example 6 is controlled by promoter methylation, colon cancer cell lines Caco-2 and HCT 116 were treated with demethylation agent, 5-aza-2′deoxycytidine (DAC, Sigma, USA) for three days at a concentration of 200 nM. Cells were harvested and total RNA was isolated from treated and untreated cell lines using Tri reagent. To determine gene expression changes by DAC treatment, transcript level between untreated and treated cell lines was directly compared. From this experiment, 280 genes were identified that show elevated expression when treated with DAC compared with the control group which was not treated with DAC. 43 common genes between the 1312 tumor repressed genes and the 280 reactivated genes were identified (FIG. 8).

### Example 8

### Confirmation of Methylation of Identified Genes

### Example 8.1

### In Silico Analysis of CpG Island in Promoter Region

The promoter regions of the 43 genes were scanned for the presence of CpG islands using MethPrimer (http://itsa.ucsf.edu/˜urolab/methprimer/index1.html). Ten genes did not contain the CpG island and were dropped from the common gene list.

### Example 8.2

### Biochemical Assay for Methylation

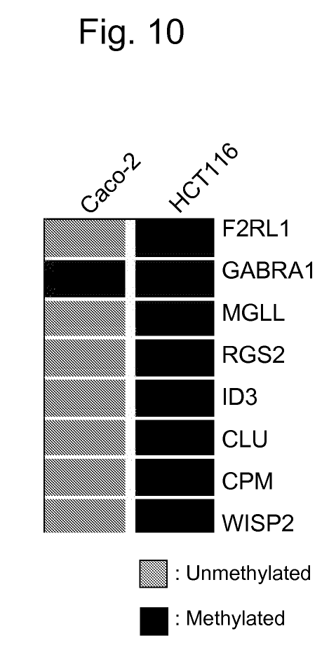

To biochemically determine the methylation status of the remaining 33 genes, methylation status of each promoter was detected using the characteristics of restriction endonucleases, HpaII (methylation-sensitive) and MspI (methylation-insensitive) followed by PCR, as well as the analytical and data interpretation method, as described in Example 3.2 above. From this, it was discovered that 25 genes were not methylated, leaving 8 confirmed candidate genes that fit the criteria of being down regulated in tumor, up regulated under demethylation conditions, contains a CpG island in its promoter and is actually methylated in the cancer cell lines. See FIGS. 9 and 10.

### Example 8.3

### Bisulfite Sequencing of Methylated Promoter

To further confirm the methylation status of the 8 additional identified genes, the inventors performed bisulfite sequencing of the individual promoters, as described in Example 3.3 above. The results confirmed that the genes were all methylated.

### Example 9

### Gene Expression Profile of the Identified Genes

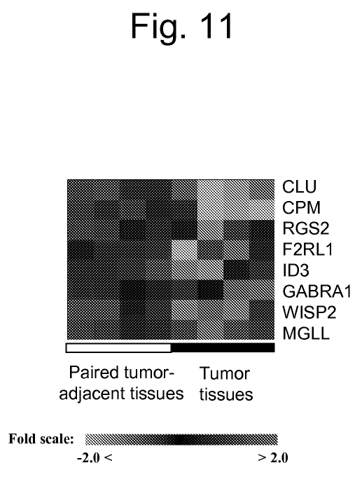

FIG. 11 shows the gene expression profiles of the 8 genes that were identified. As shown in FIG. 11, gene expression was repressed in tumor tissues compared with non-tumor tissues.

### Example 10

### Reactivation of the Additional 8 Identified Genes by Treatment with Demethylating Agent

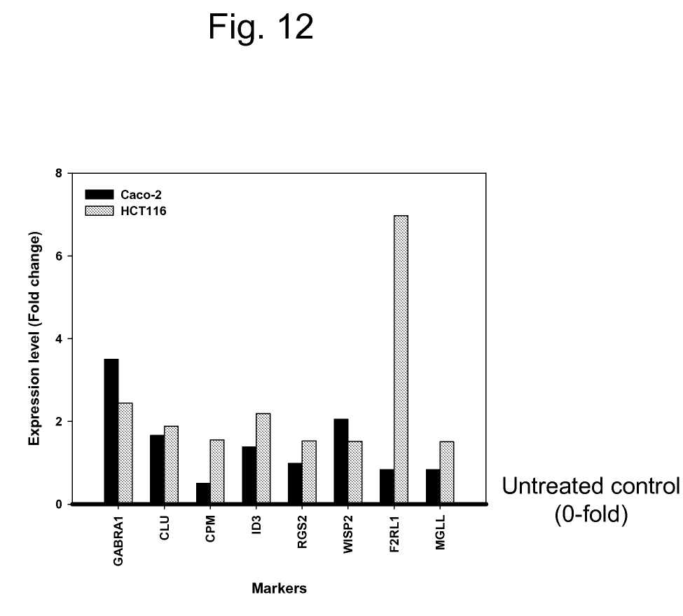

FIG. 12 shows reactivation of the additional 8 genes that were identified. As shown in FIG. 12, gene expression was reactivated in the colon cancer cells treated with demethylating agent (DAC) compared with untreated cells.

### Example 11

### Promoter Methylation Assay on Clinical Samples

To determine the clinical applicability of the methylated promoters of the 8 additionally selected genes of the present invention, methylation assay was performed with normal tissues from non-patients, and clinical samples of paired colon tumor-adjacent tissues and colon cancer tissues. Methylation assay was performed as described supra using restriction enzyme/PCR.

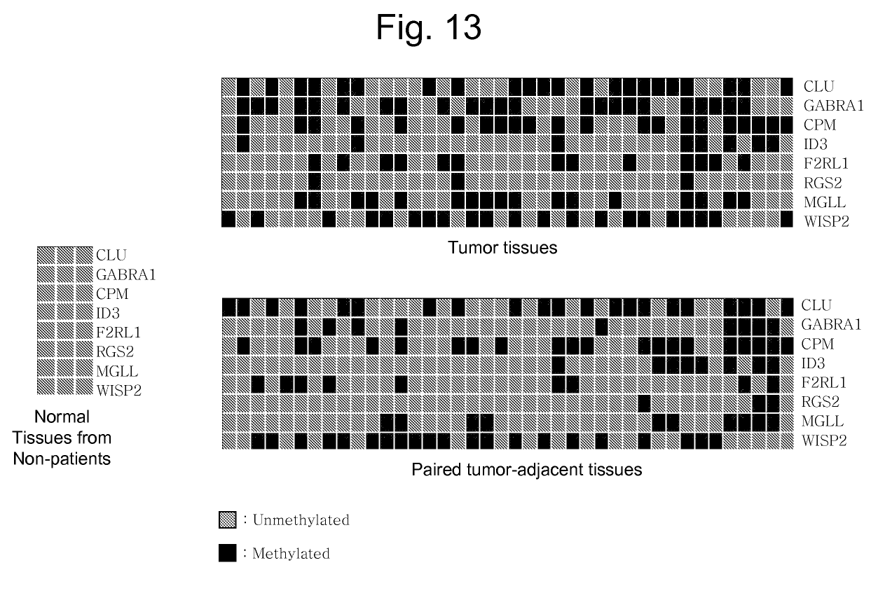

FIG. 13 shows the results of the methylation assay on normal, colon tumor, and tumor-adjacent tissue. As shown in FIG. 13, none of the genes are methylated in the normal tissues from non-patient samples (Biochain). However, all of the genes are methylated in colon cancer tissues as well as in paired tumor-adjacent tissues. All of the genes are methylated in cancer samples but not in normal cells as predicted. Moreover, as shown in FIG. 13, since the additional 8 identified genes were methylated in paired tumor-adjacent tissues, the results indicate that these 8 identified genes are useful for early detection of colon cancer.

### Example 12

### Promoter Methylation Frequency on Clinical Samples

To determine the clinical applicability of the methylated promoters of the additional 8 selected genes of the present invention, methylation assay was performed with normal tissues from non-patients, and clinical samples of paired colon tumor-adjacent tissues and colon cancer tissues. Methylation assay was performed as described supra using restriction enzyme/PCR.

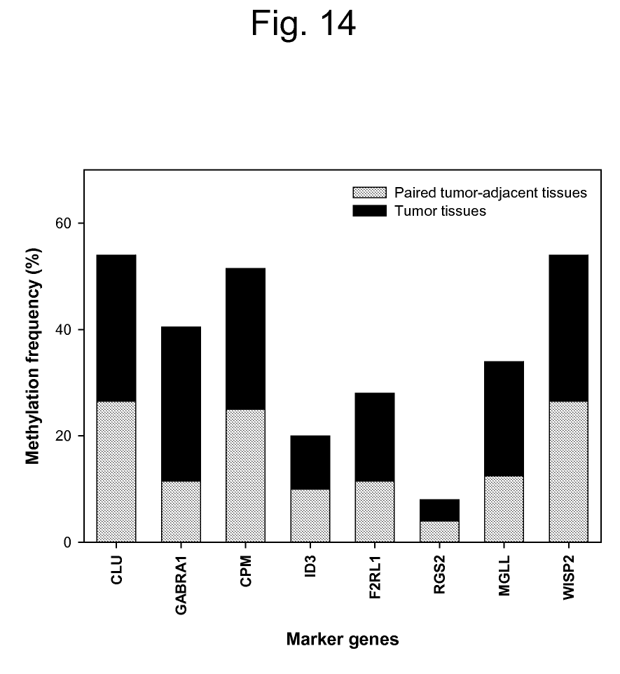

FIG. 14 shows the results of the methylation frequency on colon cancer. As shown in FIG. 14, none of the genes are methylated in the normal tissues from non-patient clinical samples (Biochain). However, all of the genes are methylated in colon cancer tissues and paired tumor-adjacent tissues. All of the genes are methylated in cancer samples but not in normal cells as predicted. As shown in FIG. 14, since the 8 additionally identified genes were methylated in paired tumor-adjacent tissues (40 samples) in addition to tumor tissues (40 samples), the results indicate that these 8 additional identified genes are useful for early detection for colon cancer.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for discovering a methylation marker gene for the conversion of a normal cell to colon cancer cell comprising:
(i) comparing converted and unconverted cell gene expression content to identify a gene that is present in greater abundance in the unconverted cell;
(ii) treating a converted cell with a demethylating agent and comparing its gene expression content with gene expression content of an untreated converted cell to identify a gene that is present in greater abundance in the cell treated with the demethylating agent; and
(iii) identifying a gene that is common to the identified genes in steps (i) and (ii), wherein the common identified gene is the methylation marker gene.

2. The method according to claim 1, comprising reviewing the sequence of the identified gene and discarding the gene for which the promoter sequence does not have a CpG island.

3. The method according to claim 1, wherein the comparing is carried out by direct comparison.

4. The method according to claim 1, wherein the comparing is carried out by indirect comparison.

5. The method according to claim 1, wherein the demethylating agent is 5 aza 2′-deoxycytidine (DAC).

6. The method according to claim 1, comprising confirming the methylation marker gene, which comprises assaying for methylation of the common identified gene in the converted cell, wherein the presence of methylation in the promoter region of the common identified gene confirms that the identified gene is the marker gene.

7. The method according to claim 6, wherein the assay for methylation of the identified gene is carried out by
i. identifying primers that span a methylation site within the nucleic acid region to be amplified,
ii. treating the genome of the converted cell with a methylation specific restriction endonuclease,
iii. amplifying the nucleic acid by contacting the genomic nucleic acid with the primers, wherein successful amplification indicates that the identified gene is methylated, and unsuccessful amplification indicates that the identified gene is not methylated.

8. The method according to claim 7, wherein the converted cell genome is treated with an isoschizomer of the methylation sensitive restriction endonuclease that cleaves both methylated and unmethylated CpG-sites as a control.

9. The method according to claim 7, wherein detecting the presence of amplified nucleic acid is carried out by hybridization with a probe.

10. The method according to claim 9, wherein the probe is immobilized on a solid substrate.

11. The method according to claim 7, wherein the amplification is carried out by PCR, real time PCR, or amplification or linear amplification using isothermal enzyme.

12. The method according to claim 1, wherein detection of methylation on the outer part of the promoter is indicative of early detection of cell conversion.

13. A method of identifying a converted colon cancer cell comprising assaying for the methylation of the marker gene identified in claim 1.

14. A method of diagnosing colon cancer or a stage in the progression of the cancer in a subject comprising assaying for the methylation of the marker gene identified using the method in claim 1.

15. The method according to claim 14, wherein the marker gene is LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1; ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1, or a combination thereof.

16. A method of diagnosing likelihood of developing colon cancer comprising assaying for methylation of a colon cancer specific marker gene in normal appearing bodily sample.

17. The method of claim 16, wherein the marker gene is LAMA2 (NT_{—}025741)—laminin alpha2(merosin, congenital); FABP4 (NT_{—}008183)—Adipocyte acid binding protein 4; GSTA2 (NT_{—}007592)—glutathione S transferase A2; STMN2 (NT_{—}008183)—Stathmin-like 2; NR4A2 (NT_{—}005403)—Nuclear receptor subfamily 4 group A, member 2; DSCR1L1 (NT_{—}007592)—Down syndrome cadidate region gene 1 like-1; AMBP (NT_{—}008470)—alpha-1-microglobulin/bikunin precursor; SEPP1 (NT_{—}006576)—selenoprotein P, plasma 1; ID3 (NT_{—}004610)—inhibitor of DNA binding 3, dominant negative helix-loop-helix protein; RGS2 (NT_{—}004487)—regulator of G-protein signalling 2; WISP2 (NT_{—}011362)—WNT1 inducible signaling pathway protein 2; MGLL (NT_{—}005612)—monoglyceride lipase; CPM (NT_{—}029419)—carboxypeptidase M 12q14.3; GABRA1 (NT_{—}023133)—gamma-aminobutyric acid (GABA) A receptor, alpha 1; CLU (NT_{—}023666)—clusterin (complement lysis inhibitor, SP-40,40, sulfated glycoprotein 2, testosterone-repressed prostate message 2, apolipoprotein J); and F2RL1 (NT_{—}006713)—coagulation factor II (thrombin) receptor-like 1, or a combination thereof.

18. The method according to claim 16, wherein the bodily sample is solid tissue, stool, or body fluids.

19. The method according to claim 16, wherein likelihood of developing colon cancer is determined by reviewing a panel of colon-cancer specific methylated genes for their level of methylation and assigning level of likelihood of developing colon cancer.
